(19) United States
(12) Patent Application Publication
(10) Pub. No.: US 2024/0299321 A1
(43) Pub. Date: Sep. 12, 2024

(54) METHODS TO SENSITIZE CANCER CELLS AND STANDARDIZED SCREENING ASSAY FOR IMMUNOTHERAPY

(71) Applicant: THE TRUSTEES OF INDIANA UNIVERSITY, Bloomington, IN (US)

(72) Inventors: Xiongbin LU, Carmel, IN (US); Xinna ZHANG, Carmel, IN (US)

(21) Appl. No.: 18/258,673

(22) PCT Filed: Dec. 10, 2021
(86) PCT No.: PCT/US2021/062842

§ 371 Date: Jun. 21, 2023

Related U.S. Application Data
(60) Provisional application No. 63/129,762, filed on Dec. 23, 2020.
(60) Provisional application No. 63/178,559, filed on Apr. 23, 2021.

(51) Int. Cl. A61K 31/167; A61K 31/4468; A61K 31/517; A61K 45/06; A61P 35/00; G01N 33/50

(52) U.S. Cl. CPC A61K 31/167; A61K 31/4468; A61K 31/517; A61K 45/06; A61P 35/00; G01N 33/505; G01N 33/5088; G01N 2500/10

(57) ABSTRACT

This invention is directed to pharmaceutical compositions and methods for increasing immunogenicity in a cancer cell by contacting a cancer cell with epigenetic inhibitors to prompt the cancer cell's production of MHC-I-associated tumor antigens.

### CROSS REFERENCE TO RELATED APPLICATIONS

This application claims priority to the following: U.S. Provisional Patent Application Nos. 63/129,762 and 63/178,559 filed on Dec. 23, 2020, and Apr. 23, 2021, respectively, the disclosure of which are expressly incorporated herein.

### INCORPORATION BY REFERENCES OF MATERIAL SUBMITTED ELECTRONICALLY

Incorporated by reference in its entirety is a computer-readable nucleotide/amino acid sequence listing submitted concurrently herewith and identified as follows: 11 kilobytes ACII (Text) file named “349712_ST25.txt,” created on Dec. 3, 2021.

### BACKGROUND OF THE DISCLOSURE

The present disclosure relates generally to a method of increasing immunogenicity in a cancer cell. More specifically, the disclosure relates to a method of contacting a cancer cell with epigenetic inhibitors to prompt the cancer cell's production of MHC-I-associated tumor antigens, resulting in the ability of an immune cell to recognize and attack the cancer cell. Further, disclosed herein are methods for generating a standardized screening assay for immunotherapy drug discovery and responses.

### SUMMARY

The present disclosure provides methods of inhibiting cancer cell growth or cancer cell proliferation by immune cells, wherein the method comprises contacting a cancer cell with an epigenetic inhibitor. While great advancements have been made in cancer immunotherapy technology, the cancer's ability to disguise itself and evade immune detection is still an issue. Described herein are methods to activate antigen processing and presentation on a cancer cell making it readily identifiable to an immune cell.

The interaction between the cancer cell and the epigenetic inhibitor augments the antigen processing activity of the cell's immunoproteasome complex and enhances the major histocompatibility class I (MHC-I)-mediated antigen presentation on the cancer cell. In essence, by contacting a cancer cell with an epigenetic inhibitor, the cancer cell begins to increase antigens presented on its surface allowing an immune cell to recognize and attack it. More particularly, in accordance with one embodiment the present disclosure describes the ability of CD8+ T-cells to recognize the cancer cell after exposure to an epigenetic inhibitor.

In some aspects, the disclosure provides methods of using epigenetic inhibitors in combination with another therapeutic molecule to increase tumor infiltration and sensitivity to CD8+ T-cell cytotoxicity. In one embodiment, a method of inhibiting cancer cell growth or cancer cell proliferation is provided, wherein the method contacting a cancer cell with an epigenetic inhibitor selected from BML-210, CUDC-101, or GSK-LSD1 in the presence of T-cells and optional checkpoint inhibitors, wherein the epigenetic inhibitor increases the cancer cell's sensitivity to CD8+ T-cell cytotoxicity. In some aspects, epigenetic inhibitor is substantially pure, is about 98% pure, is about 97% pure, is about 96%, is about 95% pure, or is about 90% pure.

In one embodiment, a method of treating a tumor with BML-210, CUDC-101, or GSK-LSD1 in combination with a therapeutic molecule is taught. In some embodiments, the therapeutic molecule is selected from known checkpoint inhibitors including for example an anti-programmed death-1 (PD-1) moiety. In some embodiments, the therapeutic molecule is an anti-cytotoxic T-lymphocyte associated protein 4 (CTLA-4) or anti-programmed death ligand 1 (PD-L1) moiety. In some embodiments, a method of treating a tumor with an epigenetic inhibitor in combination with CTLA-4 or PD-L1 inhibitor is taught. In some embodiments, the therapeutic molecule activates the T-cell.

In some embodiments, a standardized system for testing lead compounds in a laboratory is taught. In some embodiments, a personalized system using a patient's own tumor cells may be cultured and used to generate a standardized assay to determine the best treatment options with immunotherapy.

In some embodiments, a method of sensitizing a tumor cell to T-cell cytotoxicity comprises contacting the tumor cell with epigenetic inhibitors. In some embodiments, the epigenetic inhibitor is selected from BML-210, CUDC-101, or GSK-LSD1.

In some embodiments, a method is provided for identifying, and analyzing the effectiveness of compounds for their ability to enhance the sensitivity of tumor cell to T-cell cytotoxicity. In one embodiment the method comprises the steps of transplanting cancer cells into mice to generate syngeneic mammary tumors, harvesting and dissociating the resultant tumors into single cells for 2D culture, and collecting adherent cells to generate tumor organoids, treating tumor organoids having a diameter between 70 and 150 μm with a candidate drug for 48 h, contacting the treated tumor organoids with pre-activated CD8+ T cells from mice; and measuring the release of a intracellular marker (e.g., luciferase) from cells of the tumor organoids, to assess the T cell-mediated cytotoxicity effect. In one embodiment this method was used to identify BML-210, CUCU-101 and GSK-LSD1 as therapeutics for use as immunotherapy to sensitize breast cancer cells to T-cell cytotoxicity. In some embodiments, BML-210, CUCU-101 and GSK-LSD1, or any combination thereof, are used in combination with a checkpoint inhibitor to enhance the efficacy of breast cancer immune checkpoint therapy.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1A-1D shows the results of a screen of epigenetic inhibitors that enhance T cell-mediated tumor cell killing. FIG. 1A presents a Volcano plot analysis of epigenetic inhibitors' toxicity for the tumor cells in EO771 (Luc+OVA+) tumor organoids at the concentration of 1.0 μM. The compounds without significant toxicity effect in the absence of CD8+ T cells were shown in the square. FIG. 1B presents a Volcano plot analysis showing the effect of epigenetic inhibitors on the CD8+ T cell-mediated cytotoxicity in the co-culture with the EO771 tumor organoids. The EO771 tumor organoids were treated with epigenetic inhibitors for 48 h and then co-cultured with CD8+ T cells for 24 h. Compounds shown in the circle significantly promoted T cell-mediated cytotoxicity (log 2[relative viability]<−0.5; p<0.05). Relative viability=(tumor cell viability of treated group)/(tumor cell viability of control group). FIG. 1C presents a Volcano plot analysis showing the effect of epigenetic inhibitors on the CD8+ T cell-mediated cytotoxicity for the EO771 (Luc+OVA+) cells in 2D culture. Compounds shown in the circle significantly promoted T cell cytotoxicity (log 2[relative viability]<−0.5; p<0.05). The cytotoxicity measured in graphs of FIGS. 1B and 1C was quantified using luciferase assay. FIG. 1D presents a Volcano plot analysis showing the effect of epigenetic inhibitors on OVA antigen presentation of the EO771 (Luc+OVA+) cells. Compounds shown in the rectangle significantly promoted OVA antigen expression (log 2[OVA presentation/control]>0.5; p<0.05). 10,000 events per cell sample in flow cytometry were collected for data analysis. The mean fluorescence intensity (MFI) from the cells stained with APC-conjugated OVA peptide SIINFEKL (SEQ ID NO: 55) antibody represents the OVA expression levels.

FIGS. 2A-2D: Validation of anti-tumor activity of the three drug candidates in mouse and human tumor organoids. FIG. 2A is a graph of the size of OVA+ EO771 tumor organoids treated with control or drug candidates in the co-culture with OVA-specific CD8+ T cells. Optical images were analyzed using ImageJ to determine the organoid size. FIG. 2B is a graph presenting the death proportion of the OVA+ EO771 tumor cells in the tumor organoids treated with control or drug candidate, which was measured by flow cytometry analysis. The SYTOX Blue reagent was used to determine the cell death proportion. FIG. 2C is a graph presenting the death proportion of NY-ESO-1+ MDA-MB-468 cells treated with control or drug candidates in the co-culture with the NY-ESO-1-specific CD8+ T cells. SYTOX Blue reagent was used to determine the cell death proportion. FIG. 2D provides the percentages of the NY-ESO-1⁺ CD8+ T cells positive for GZMB, IFNγ and TNF-α in the co-culture with the MDA-MF-468 tumor organoids treated with control or drug candidates. For statistical analysis of data, one-way ANOVA test was used in (FIG. 2A) and two-way ANOVA test was used in (FIGS. 2B-2C). Data are presented as mean±SEM and are representative of 3 parallel experiments. **, p<0.01; ***, p<0.001; ****, p<0.0001; ns, no significance.

FIGS. 3A-3I: Anti-tumor activity of drug candidates in mouse breast tumor models. FIG. 3A presents a schematic of the drug treatment of mouse breast tumor models. Mammary tumors were harvested and the tumor growth (FIG. 3B) and weight (FIG. 3C) of the EO771 tumors from the tumor-bearing C57BL/6 mice treated with vehicle control, BML-210 (20 mg kg⁻¹), or CUDC-101 (20 mg kg⁻¹) were determined. FIG. 3D presents the proportions of total T, CD4⁺ T, and CD8⁺ T cells in total immune (CD45⁺) cells in the EO771 tumors treated with control, BML-210 or CUDC-101. FIG. 3E presents the percentage of active cells in total CD8⁺ T cells, indicated by GZMB⁺, IFNγ⁺, TNFα⁺ in flow cytometry analysis. Levels of secreted IFNγ and TNF-α from CD8+ T cells isolated from the tumor tissues with and without PMA and ionomycin stimulation is shown in FIGS. 3F and 3G, respectively. Gross dissected mammary EO771 tumors from the tumor-bearing immunodeficient nude mice showed no significant difference in size between those treated with vehicle control, BML-210 (20 mg kg⁻¹), or CUDC-101 (20 mg kg⁻¹). Tumors from immunodeficient mice were harvested at day 18 post injection. The tumor growth of the EO771 tumors from the tumor-bearing C57BL/6 mice treated with isotype control, CUDC-101 (20 mg kg⁻¹), BML-210 (20 mg kg⁻¹), anti-CD8 (10 mg kg⁻¹)+CUDC-101 (20 mg kg⁻¹), anti-CD8 (10 mg kg⁻¹)+BML-210 (20 mg kg⁻¹), anti-CD4 (10 mg kg⁻¹)+CUDC-101 (20 mg kg⁻¹) or anti-CD4 (10 mg kg⁻¹)+BML-210 (20 mg kg⁻¹) are shown in FIGS. 3H and 3I. Tumors from tumor-bearing C57BL/6 mice were harvested at day 28 post injection. For statistical analysis of data, one-way ANOVA test was used in (FIGS. 3B and 3C), and two-way ANOVA test was used in (FIGS. 3D-3I). Data are presented as mean±SEM. *, p<0.05; **, p<0.01; ***, p<0.001; ****, p<0.0001; ns, no significance.

FIGS. 4A-4G: BML-210 treatment up-regulates tumor antigen processing and presentation. FIGS. 4A and 4B are bar graphs showing the BML-210-induced upregulation of differentially expressed genes (DEGs) in antigen processing and presentation in EO771 cells (FIG. 4A) and human MDA-MB-468 cells (FIG. 4B) treated with control or BML-210 (1.0 μM) as measured by quantitative RT-PCR. FIG. 4C shows the levels of H-2Kb on the OVA+ EO771 cells treated with control or BML-210, which were determined by mean fluorescence intensity (MFI) in flow cytometry analysis. FIG. 4D shows the levels of HLA-A,B,C on the NY-ESO-1+ MDA-MB-468 cells treated with control or BML-201, determined by MFI in flow cytometry analysis. Confocal images where taken and quantitated for H-2Kb and HLA-A2 antigen presented on the OVA+ EO771 (FIG. 4E) and NY-ESO-1+ cells (FIG. 4F) treated with control or BML-210. FIG. 4G shows the death proportions of the OVA+ EO771 tumor cells with (using shB2M) and without B2M knockdown in the tumor organoids as measured by flow cytometry analysis. The SYTOX Blue reagent was used to determine the cell death proportion. One-way ANOVA test was conducted for statistical analysis. Data are presented as mean±SEM and are representative of 3 independent experiments. *, p<0.05; **, p<0.01; ***, p<0.001; ****, p<0.0001; ns, no significance.

FIG. 5A-5F: BML-210 treatment enhances anti-tumor response in combination with PD-1 blockade. FIGS. 5A and 5B show the quantitative analysis of CD8+ T cells (FIG. 5A) or PMN-MDSCs (CD11b⁺Ly6G⁺Ly6C^{low}F4/80⁻; FIG. 5B) in mouse EO771 tumors implanted in C57BL/6 mice and treated with control or BML-210. FIGS. 5C and 5D provide the tumor growth (FIG. 5C) and weight (FIG. 5D) of the EO771 tumors from the tumor-bearing C57BL/6 mice treated with vehicle control, BML-210 (20 mg kg⁻¹), PD-1 antibody (200 μg per mouse), or BML-210 and PD-1 antibody combo. FIGS. 5E and 5F show the quantitative results of CD8⁺ T cells and cleaved Caspase 3⁺ cells in the tumor tissues with indicated treatment. One-way ANOVA test was conducted for statistical analysis in FIGS. 5A-5D, and two-way ANOVA was used in FIGS. 5E and 5F. A total of 20 images in each group were analyzed to quantify the cell proportion. All the data are presented as mean±SEM. *, p<0.05; **, p<0.01; ***, p<0.001; ****, p<0.0001; ns, no significance.

FIG. 6: Treatment of BML-210, CUDC-210 or GSK-LSD1 enhances tumor antigen presentation and the efficiency of immune checkpoint blockade therapy. FIG. 6 shows the death proportions of tumor cells in PDOs from six breast cancer patients treated with control, BML-201, CUDC-210 or GSK-LSD1 after co-culture with autologous CD8+ T cells. Two-way ANOVA test was used for statistical analysis and data are presented as mean±SEM. ***, p<0.001; ****, p<0.0001; ns, no significance.

### DETAILED DESCRIPTION

### Definitions

In describing and claiming the invention, the following terminology will be used in accordance with the definitions set forth below.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein the term “pharmaceutically acceptable salt” refers to salts of compounds that retain the biological activity of the parent compound, and which are not biologically or otherwise undesirable. Many of the compounds disclosed herein are capable of forming acid and/or base salts by virtue of the presence of amino and/or carboxyl groups or groups similar thereto.

As used herein, the term “treating” includes alleviation of the symptoms associated with a specific disorder or condition and/or preventing or eliminating said symptoms. “Inhibition” of disease progression or disease complication in a subject means preventing or reducing the disease progression and/or disease complication in the subject.

As used herein an “effective” amount or a “therapeutically effective amount” of drug refers to a nontoxic but sufficient amount of drug to provide the desired effect. The amount that is “effective” will vary from subject to subject, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

As used herein, the term “purified” and like terms relate to the isolation of a molecule or compound in a form that is substantially free of contaminants normally associated with the molecule or compound in a native or natural environment. As used herein, the term “purified” does not require absolute purity; rather, it is intended as a relative definition.

The term “isolated” requires that the referenced material be removed from its original environment (e.g., the natural environment if it is naturally occurring). For example, a naturally-occurring nucleic acid present in a living animal is not isolated, but the same nucleic acid, separated from some or all of the coexisting materials in the natural system, is isolated.

As used herein the term “patient” without further designation is intended to encompass any warm blooded vertebrate domesticated animal (including for example, but not limited to livestock, horses, mice, cats, dogs and other pets) and humans receiving a therapeutic treatment whether or not under the supervision of a physician.

“Subject” can refer to living organisms such as mammals, including, but not limited to humans, livestock, dogs, cats, and other mammals. Administration of the tocotrienol can be carried out at dosages and for periods of time effective for treatment of a subject. In some embodiments, the subject is a human. In some embodiments, the pharmacokinetic profiles of the systems of the present invention are similar for male and female subjects.

As used herein the term “epigenetic inhibitor” defines any compound that modulates the activity of a methyltransferase, demethylase, histone acetyltransferase (HAT), histone deacetylase (HDAC), or acetylated lysine reader protein.

As used herein, the term “sensitizing”, in reference to a cancer cell, defines a process wherein a cancer cell is induced by an agent to increase the production and/or display of antigens in an amount that is more easily recognized by a CD8+ T-cell relative to the cancer cell prior to contact with the sensitizing agent. Examples of sensitizing agents include epigenetic inhibitors, such as BML-210, CUDC-101, and GSK-LSD1.

### EMBODIMENTS

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

In some embodiments, a method of inhibiting cancer cell proliferation or growth is provided wherein the method comprises contacting a cancer cell with a small molecule, wherein the small molecule is an epigenetic inhibitor, optionally wherein the small molecule comprises BML-210, CUDC-101, and/or GSK-LSD1, and the cancer cell becomes more sensitized to the cytotoxic effects of a CD8+ T-cell. In some embodiments, the cancer cell is a form of breast cancer.

BML-210 is available for purchase from commercial vendors for research purposes. For example, BML-210 may be purchased from Enzo Life Sciences. BML-210 has a chemical formula of C₂₀H₂₅N₃O₂. One understood structure of BML-210 is

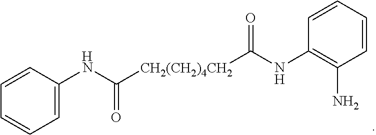

CUDC-101 is available for purchase from commercial vendors for research purposes. For example, CUDC-101 may be purchased from Sellekchem. CUDC-101 has a chemical formula of C₂₄H₂₆N₄O₄. One understood structure of CUDC-101 is

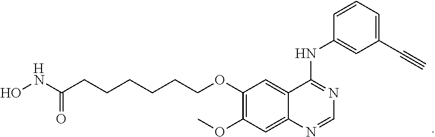

GSK-LSD1 is available for purchase from commercial vendors for research purposes. GSK-LSD1 has a chemical formula of C14H20N2. One understood structure of GSK-LSD1 is

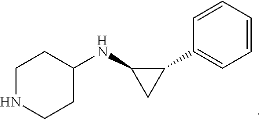

In some embodiments, the epigenetic inhibitor is provide alone. In some embodiments, the epigenetic inhibitor is substantially pure, about 98% pure, about 97% pure, about 96% pure, about 95% pure, about 94% pure, about 93% pure, about 92% pure, about 91% pure, or about 90% pure.

In some embodiments, the small molecule is provided in combination with other therapeutics, antibodies, or small molecules. In some embodiments, the other therapeutics include PD-1, PD-L1, or CTLA-4 inhibitors. In these embodiments, the PD-1, PD-L1, or CTLA-4 inhibitors are useful, in part, to help the CD8+ T-cell destroy the cancer cell.

In some embodiments, the cancer cell is capable of forming a tumor. In some embodiments, the cancer cell is from a breast cancer.

In accordance with one embodiment a method of enhancing the efficacy of CD8+ T cell cytotoxicity against a cancerous cell is provide wherein the method comprises contacting said cancerous cell with an epigenetic inhibitor. In one embodiment the epigenetic inhibitor is selected from the group consisting of BML-210, CUDC-101 and GSK-LSD1 or combinations thereof. In one embodiment the method further comprises a step of administering a checkpoint inhibitor in conjunction with the administration of the epigenetic inhibitor. In one embodiment the checkpoint inhibitor is administer simultaneously with the epigenetic inhibitor. In another embodiment the checkpoint inhibitor is administered prior to or after administration of the epigenetic inhibitor, wherein the duration of time between the administration of the checkpoint inhibitor and the epigenetic inhibitor is sufficiently brief to allow for a therapeutic effect of the co-administration of the two inhibitors. In one embodiment the two inhibitors are administered within 1, 2, 3, 4, 8, 12, 24 or 48 hours of one another.

In one embodiment the checkpoint inhibitor administered in conjunction with the epigenetic inhibitor is an inhibitor of cytotoxic T-lymphocyte associated protein 4 (CTLA-4), programmed cell death 1 (PD-1) or programmed death ligand 1 (PD-L1). In one embodiment the CTLA-4 inhibitor is an antibody that specifically binds to CTLA-4, optionally wherein the antibody is Ipilimumab. In one embodiment the PD-1 inhibitor is an antibody that specifically binds to PD-1, optionally wherein the antibody is Nivolumab or pembrolizumab. In one embodiment the PD-L1 inhibitor is an antibody that specifically binds to PD-L1, optionally wherein the antibody is atezolizumab. In one embodiment a combination of any of the inhibitors of CTLA-4, PD-1 and PD-L1 are used in conjunction with one or more epigenetic inhibitors to form a pharmaceutical composition for contact with cancer cells to enhance the efficacy of CD8+ T cell cytotoxicity against cancerous cells contacted with the composition. In one embodiment the cancerous cells are breast cancer cells, optionally wherein the cancer cells are part of a solid tumor.

In one embodiment a pharmaceutical composition is provided comprising an epigenetic inhibitor selected from the group consisting of BML-210, CUDC-101 and GSK-LSD1, a checkpoint inhibitor, and a pharmaceutically acceptable carrier. In one embodiment the checkpoint inhibitor of the composition is an inhibitor of CTLA-4, PD-1 or PD-L1. In one embodiment the checkpoint inhibitor of the composition is selected from the group consisting of atezolizumab, Ipilimumab, Nivolumab and pembrolizumab.

In accordance with one embodiment a method of increasing the presentation of antigen-loaded MHC-I complex on a cancer cell is provided, wherein the method comprises contacting the cancer cell with an epigenetic inhibitor selected from the group consisting of BML-210, CUDC-101 and GSK-LSD1, optionally wherein the cancer cell is a breast cancer cell. In one embodiment the method is conducted in vivo in a patient afflicted with cancer, wherein the cancer cells are contacted in vivo with an epigenetic inhibitor selected from the group consisting of BML-210, CUDC-101 and GSK-LSD1 and optionally in conjunction with administration of a checkpoint inhibitor.

In one embodiment a method of inhibiting cancer cell growth or cancer cell proliferation is provided. The method comprises contacting a cancer cell with an epigenetic inhibitor selected from BML-210, CUDC-101, or GSK-LSD1 in the presence of T-cells. In one embodiment cancer cell growth or cancer cell proliferation is inhibited in a cancer patient by administering a pharmaceutical composition comprising an epigenetic inhibitor selected from BML-210, CUDC-101, or GSK-LSD1 and a pharmaceutically acceptable carrier. In one embodiment the cancer cell are part of a solid tumor, optionally wherein the cancer is breast cancer. In a further embodiment the method of inhibiting cancer cell growth or cancer cell proliferation comprises the administration of a checkpoint inhibitor, optionally wherein the checkpoint inhibitor is an inhibitor of cytotoxic T-lymphocyte associated protein 4 (CTLA-4) or programmed death ligand 1 (PD-L1).

In one embodiment a method of for identifying compounds that enhance the sensitivity of tumor cell to T-cell cytotoxicity is provided. In one embodiment the method comprises
generating tumor organoids from cancer cells;
treating said generated tumor organoids that have a diameter between 70 and 150 μm with a candidate drug for 48 h;
contacting the treated tumor organoids with pre-activated CD8+ T cells from mice; and
measuring the release of a intracellular marker from cells of the tumor organoids, to assess the T cell-mediated cytotoxicity effect. Optionally the intracellular marker is luciferase. In one embodiment the candidate drug is an epigenetic inhibitor. In one embodiment the method further comprises contacting the tumor organoids with a checkpoint inhibitor, optionally wherein the checkpoint inhibitor is an inhibitor of cytotoxic T-lymphocyte associated protein 4 (CTLA-4) or programmed death ligand 1 (PD-L1).

In some aspects, the disclosure provides methods of using epigenetic inhibitors in combination with another therapeutic molecule to increase tumor infiltration and sensitivity to CD8+ T-cell cytotoxicity. In one embodiment, a method of inhibiting cancer cell growth or cancer cell proliferation is provided, wherein the method comprises contacting a cancer cell with an epigenetic inhibitor selected from BML-210, CUDC-101, or GSK-LSD1 in the presence of T-cells and optionally checkpoint inhibitors, wherein the epigenetic inhibitor increases the cancer cell's sensitivity to CD8+ T-cell cytotoxicity. In some aspects, epigenetic inhibitor is substantially pure, is about 98% pure, is about 97% pure, is about 96%, is about 95% pure, or is about 90% pure.

In one embodiment, a method of treating a tumor with BML-210, CUDC-101, or GSK-LSD1 in combination with a therapeutic molecule is taught. In some embodiments, the therapeutic molecule is selected from known checkpoint inhibitors including for example an anti-programmed death-1 (PD-1) moiety. In some embodiments, the therapeutic molecule comprises is an anti-cytotoxic T-lymphocyte associated protein 4 (CTLA-4) or anti-programmed death ligand 1 (PD-L1) moiety. In some embodiments, a method of treating a tumor with an epigenetic inhibitor in combination with CTLA-4 or PD-L1 inhibitor is taught.

In accordance with the present disclosure a drug screen platform was used to identify epigenetic inhibitors that increase a cancer cell's sensitivity to CD8+ T-cell cytotoxicity. In one embodiment the drug screen platform is composed of breast tumor organoids and tumor-specific CD8+ T cells. A lentiviral vector expressing OVA was transduced to the Luc+EO771 cells. The resulting OVA+ cells were orthotopically transplanted to C57BL/6 mice to generate syngeneic mammary tumors. The tumors were harvested and dissociated to single cells for 2D culture and only the adherent cells were collected to generate tumor organoids. 2 ml of tumor organoid culture medium (2×10⁵ cells per ml) were seeded into 6-well culture plate with ultra-low attachment surface. After 7-day culture, tumor organoids were filtered by cell strainers with nylon mesh between 70 and 150 μm. The tumor organoids with diameter between 70 and 150 μm were treated with drugs for 48 h in the matrigel-free medium. The treated tumor organoids were then co-cultured with the pre-activated and OVA-specific CD8+ T cells from OT-I mice in the breast organoid culture medium containing 10 ng ml-1 IL2 without matrigel for 24 h. The luciferase released from the EO771 cells was measured using a Dual-luciferase report assay system (Promega) on a BioTek Cytation5 imaging reader, to assess the T cell-mediated cytotoxicity effect.

Organoid diameter versus cell seeding concentration at day 2 and day 7 of organoid culture were measured. Optical images tumor organoids at day 7 were analyzed using ImageJ to determine the organoid size.

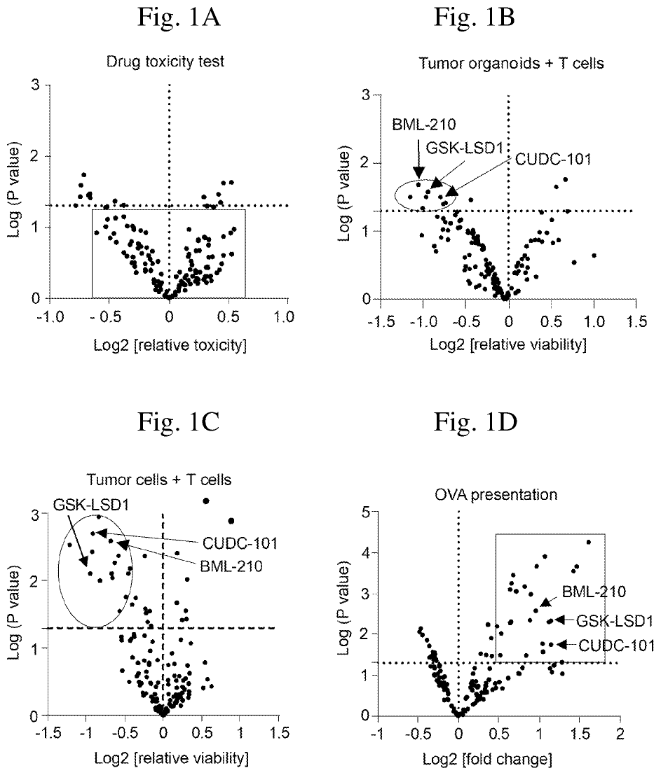

FIG. 1A shows a volcano plot analysis of epigenetic inhibitors' toxicity for the tumor cells in EO771 (Luc+OVA+) tumor organoids at the concentration of 1.0 μM. The compounds without significant toxicity effect in the absence of CD8+ T cells were shown in the square. FIG. 1B shows a volcano plot analysis showing the effect of epigenetic inhibitors on the CD8+ T cell-mediated cytotoxicity in the co-culture with the EO771 tumor organoids. The EO771 tumor organoids were treated with epigenetic inhibitors for 48 h and then co-cultured with CD8+ T cells for 24 h. Compounds shown in the circle significantly promoted T cell-mediated cytotoxicity (log 2[relative viability]<−0.5; p<0.05). Relative viability=(tumor cell viability of treated group)/(tumor cell viability of control group). FIG. 1C shows a volcano plot analysis showing the effect of epigenetic inhibitors on the CD8+ T cell-mediated cytotoxicity for the EO771 (Luc+OVA+) cells in 2D culture. Compounds shown in the circle significantly promoted T cell cytotoxicity (log 2[relative viability]<−0.5; p<0.05). The cytotoxicity in (FIG. 1B) and (FIG. 1C) was quantified using a luciferase assay. FIG. 1D shows a volcano plot analysis showing the effect of epigenetic inhibitors on OVA antigen presentation of the EO771 (Luc+OVA+) cells. Compounds shown in the rectangle significantly promoted OVA antigen expression (log 2[OVA presentation/control]>0.5; p<0.05). 10,000 events per cell sample in flow cytometry were collected for data analysis. The mean fluorescence intensity (MFI) from the cells stained with APC-conjugated OVA peptide SIINFEKL (SEQ ID NO: 55) antibody represents the OVA expression levels. One-way ANOVA test was conducted in FIG. 1A-1D. p value <0.05 in the volcano plots was considered as significant difference. The data for the drug screen represent 3 parallel experiments.

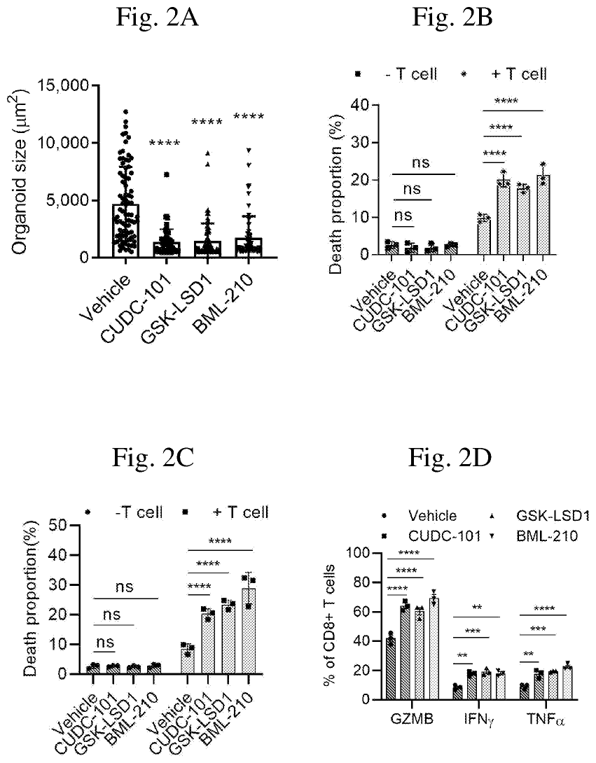

FIG. 2A shows the size of OVA+ EO771 tumor organoids treated with control or drug candidates in the co-culture with OVA-specific CD8+ T cells. Optical images were analyzed using ImageJ to determine the organoid size. FIG. 2B shows the death proportion of the OVA+ EO771 tumor cells in the tumor organoids treated with control or drug candidate, which was measured by flow cytometry analysis. The SYTOX Blue reagent was used to determine the cell death proportion. The percentages of OVA-specific CD8+ T cells positive for GZMB, IFNγ and TNF-α in the co-culture with tumor organoids treated with control or drug candidates was measured. The secretion of IFNγ and TNF-α was stimulated with PMA and ionomycin. A drug validation test using NY-ESO-1+ MDA-MB-468 organoids co-cultured with NY-ESO-1-specific CD8⁺ T cells. Human breast cancer associated fibroblasts (CAFs) were used with MDA-MB-468 cells to generate tumor organoids. FIG. 2C shows the death proportion of NY-ESO-1+ MDA-MB-468 cells treated with control or drug candidates in the co-culture with the NY-ESO-1-specific CD8+ T cells. SYTOX Blue reagent was used to determine the cell death proportion. FIG. 2D shows the percentages of the NY-ESO-1+ CD8+ T cells positive for GZMB, IFNγ and TNF-α in the co-culture with the MDA-MF-468 tumor organoids treated with control or drug candidates. For statistical analysis of data, one-way ANOVA test was used in FIG. 2A and two-way ANOVA test was used in FIGS. 2B, 2C and 2D. Data are presented as mean±SEM and are representative of 3 parallel experiments. *, p<0.05; **, p<0.01; ****, p<0.0001

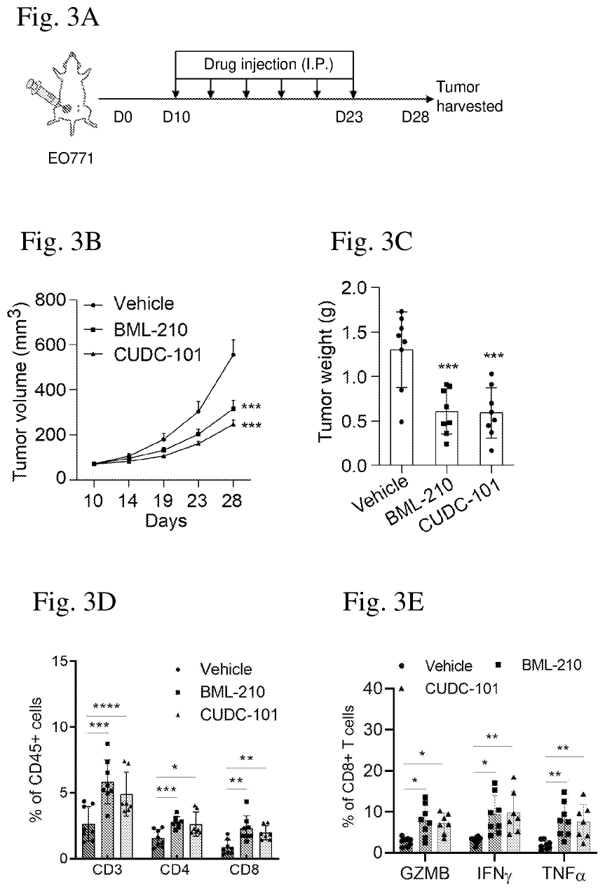

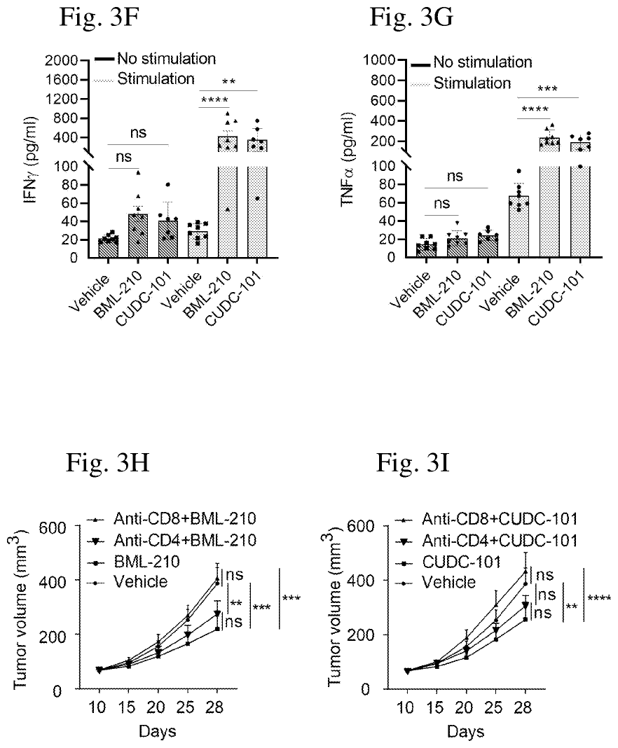

FIG. 3A shows a drug treatment scheme of mouse breast tumor models. FIGS. 3B and 3C show the tumor growth (FIG. 3B) and weight (FIG. 3C) of the EO771 tumors from the tumor-bearing C57BL/6 mice treated with vehicle control, BML-210 (20 mg kg-1), or CUDC-101 (20 mg kg-1). FIG. 3D shows the proportions of total T, CD4+ T, and CD8+ T cells in total immune (CD45+) cells in the EO771 tumors treated with control, BML-210 or CUDC-101. FIG. 3E shows a percentage of active cells in total CD8+ T cells, indicated by GZMB+, IFNγ+, TNFα+ in flow cytometry analysis. IHC staining of Ki67+ and cleaved Caspase 3+ cells in the tumor tissues was conducted and. FIGS. 3F and 3G show the quantitative results. The tumor growth of the EO771 tumors from the tumor-bearing C57BL/6 mice treated with isotype control, CUDC-101 (20 mg kg-1), BML-210 (20 mg kg-1), anti-CD8 (10 mg kg-1)+CUDC-101 (20 mg kg-1), anti-CD8 (10 mg kg-1)+BML-210 (20 mg kg-1), anti-CD4 (10 mg kg-1)+CUDC-101 (20 mg kg-1) or anti-CD4 (10 mg kg-1)+BML-210 (20 mg kg-1) is shown in FIGS. 3H and 3I. Tumors from tumor-bearing C57BL/6 mice were harvested at day 28 post injection. For statistical analysis of data, one-way ANOVA test was used in FIGS. 3B, 3C, 3F and 3G, and two-way ANOVA test was used in FIGS. 3D, 3E, 3H and 3I. Data are presented as mean±SEM. *, p<0.05; **, p<0.01; ***, p<0.001; ****, p<0.0001; ns, no significance.

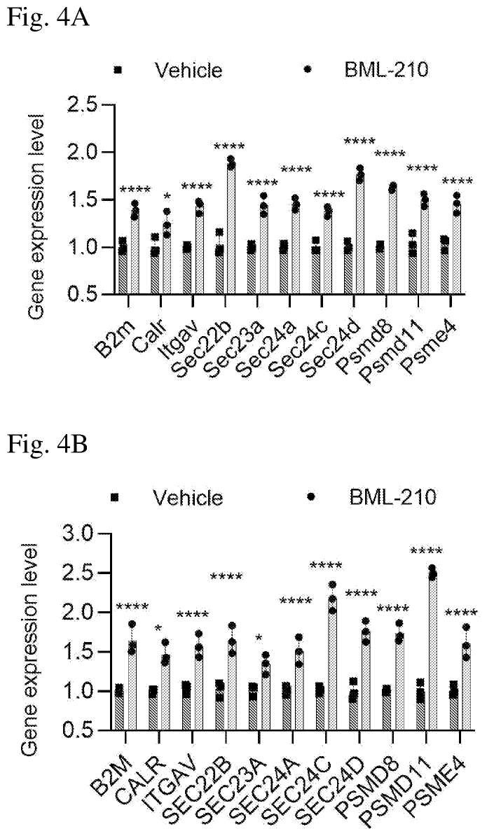

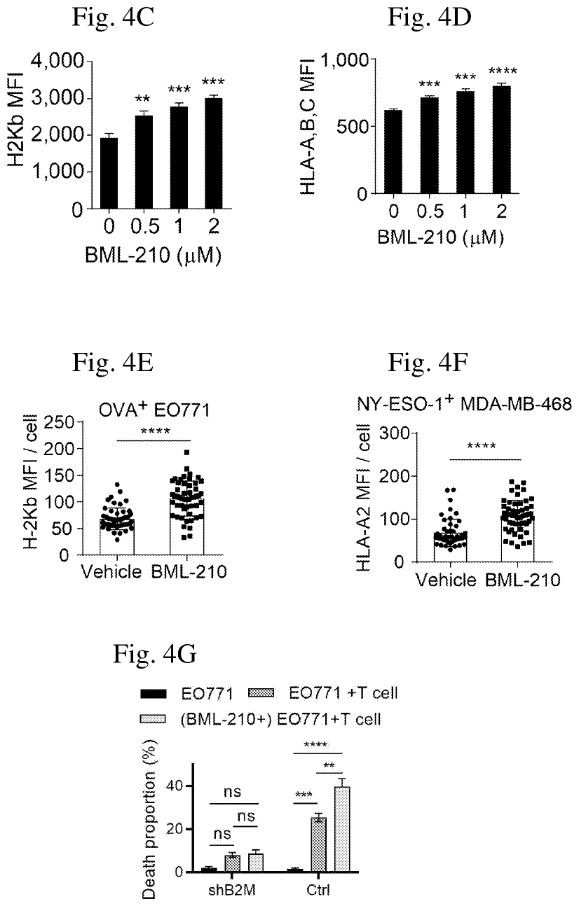

FIG. 4A and FIG. 4B show validation of up-regulated genes in mouse EO771 and human MDA-MB-468 cells, respectively, treated with control or BML-210 (1.0 NM) as measured by quantitative RT-PCR. FIG. 4C shows levels of H-2Kb on the OVA+ EO771 cells treated with control or BML-210, which were determined by mean fluorescence intensity (MFI) in flow cytometry analysis. FIG. 4D shows levels of HLA-A,B,C on the NY-ESO-1+ MDA-MB-468 cells treated with control or BML-201, determined by MFI in flow cytometry analysis. FIG. 4E and FIG. 4F show a quantitative analysis of confocal images for assessing H-2Kb and HLA-A2 antigen presentation. FIG. 4G shows the death proportions of the OVA+ EO771 tumor cells with and without B2M knockdown in the tumor organoids were measured by flow cytometry analysis. The SYTOX Blue reagent was used to determine the cell death proportion. One-way ANOVA test was conducted for statistical analysis. Data are presented as mean±SEM and are representative of 3 independent experiments. *, p<0.05; **, p<0.01; ***, p<0.001; ****, p<0.0001.

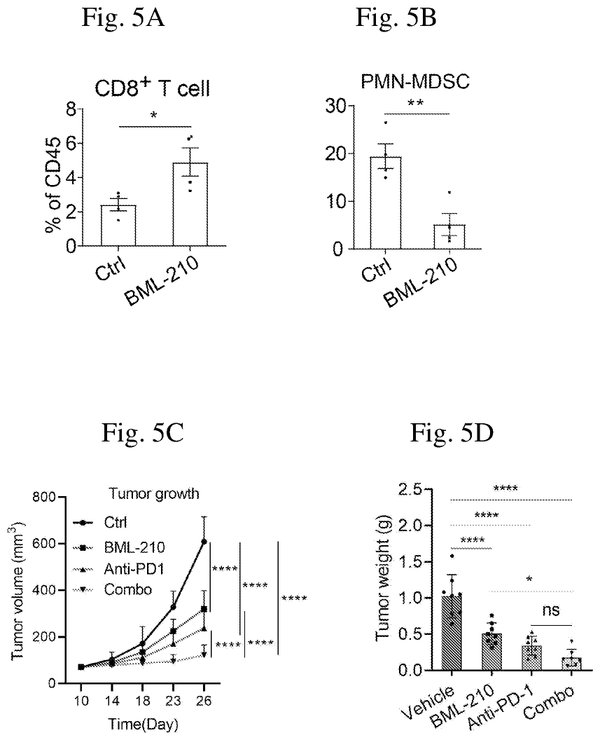

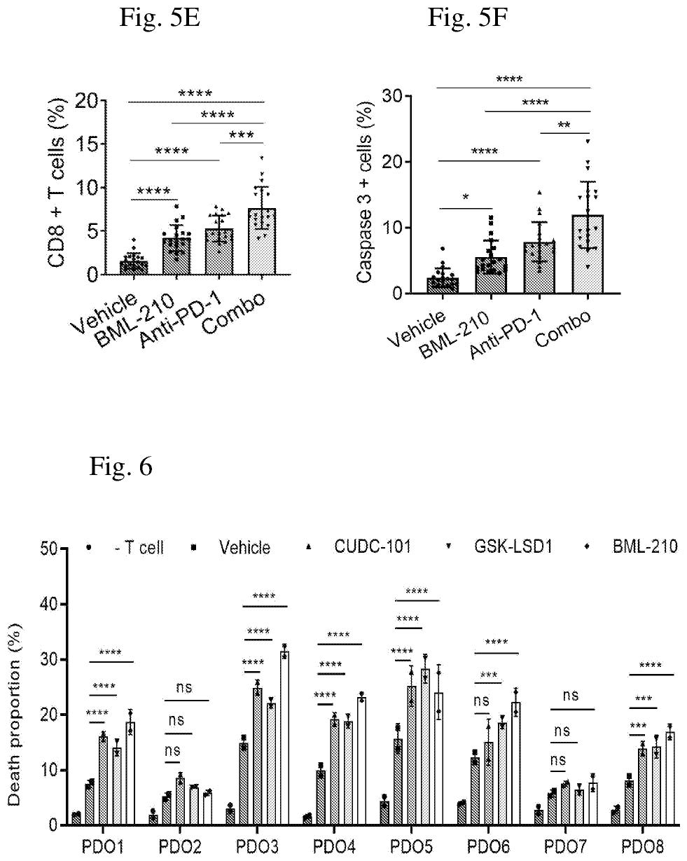

An analysis of immune profile and microenvironment of the mouse EO771 tumors in C57BL/6 mice treated with control or BML-210 using a CyTOF panel containing 26 markers (Table 1) was conducted. tSNE representation of the immune cell subtypes and percentages of distinct immune cell populations in the tumors were assessed. FIGS. 5A and 5B show tSNE representation and quantitative analysis of CD8+ T cells (FIG. 5A) and PMN-MDSCs (CD11b⁺Ly6G⁺Ly6ClowF4/80⁻) (FIG. 5B) in the EO771 tumors with control or BML-210 treatment. FIGS. 5C and 5D show the tumor growth and weight of the EO771 tumors from the tumor-bearing C57BL/6 mice treated with vehicle control, BML-210 (20 mg kg-1), PD-1 antibody (200 μg per mouse), or BML-210 and PD-1 antibody combo. FIGS. 5E and 5F show the quantitative results of CD8+ T cells and cleaved Caspase 3+ cells in the tumor tissues with indicated treatment. One-way ANOVA test was conducted for statistical analysis in (FIGS. 5A-5D), and two-way ANOVA was used in (FIGS. 5E and 5F). Each individual symbol in FIGS. 5E and 5F represents the percentage of the positive cells from the total cells in an independent field. A total of 20 images in each group were analyzed to quantify the cell proportion. All the data are presented as mean±SEM. *, p<0.05; **, p<0.01; ***, p<0.001; ****, p<0.0001; ns, no significance.

TABLE 1
Antibody panel for Cy TOF analysis

| | Marker | Clone | Tag | Cat. # | Sample (μl) |
|---|---|---|---|---|---|
| 1 | CD45 | 30- F11 | 89Y | 3089005B | 0.25 |
| 2 | I-A/I-E | M5/114.15.2 | 209Bi | 3209006B | 0.5 |
| 3 | CD274/PD-L1 | 10F.9G2 | 153Eu | 3153016B | 0.25 |
| 4 | CD3e | 145-2C11 | 152Sm | 3152004B | 0.25 |
| 5 | CD8a | 53-6.7 | 146Nd | 3146003B | 0.5 |
| 6 | CD25 | 3C7 | 150Nd | 3150002B | 0.25 |
| 7 | CD127/IL7Ra | A7R34 | 175Lu | 3175006B | 0.5 |
| 8 | CD62L | MEL-14 | 160Gd | 3160008B | 0.17 |
| 9 | CD69 | H1.2F3 | 145Nd | 3145005B | 0.5 |
| 10 | TCRb | H57-597 | 143Nd | 3143010B | 0.5 |
| 11 | CD44 | IM7 | 171Yb | 3171003B | 0.2 |
| 12 | CD11b | M1/70 | 148Nd | 3148003B | 0.17 |
| 13 | CD11c | N418 | 142^{Nd} | 3142003B | 0.5 |
| 14 | F4/80 | BM8 | 159Tb | 3159009B | 0.25 |
| 15 | Ly-6C | HK1.4 | 162Dy | 3162014B | 0.2 |
| 16 | Ly-6G | 1A8 | 141Pr | 3141008B | 0.5 |
| 17 | NK1.1 | PK136 | 170Er | 3170002B | 0.5 |
| 18 | CD45R/B220 | RA3-6B2 | 176Yb | 3176002B | 0.5 |
| 19 | CD19 | 6D5 | 149Sm | 3149002B | 0.5 |
| 20 | TER-119 | TER-119 | 154Sm | 3154005B | 0.25 |
| 21 | EpCAM | G8.8 | 166Er | 3166014B | 0.25 |
| 22 | Vimentin | RV202 | 156Gd | 315602A | 0.25 |
| 23 | CCR7 | 4B12 | 164Dy | 3164013A | 0.25 |
| 24 | LAG3 | C9B7W | 174Yb | 3174019B | 0.25 |
| 25 | CD4 | RM4-5 | 172Yb | 3172003B | 0.5 |
| 26 | CD206 | C068C2 | 169Tm | 3169021B | 0.25 |

FIG. 6 shows the death proportions of tumor cells in the PDOs from breast cancer patients treated with control, BML-201, CUDC-210 or GSK-LSD1 after co-culture with autologous CD8+ T cells. Two-way ANOVA test was used for statistical analysis and data are presented as mean±SEM. *, p<0.05; **, p<0.01; ns, no significance.

The disclosure will be more fully understood upon consideration of the following non-limiting Examples.

### Example 1 Methods

Cell lines: Mouse breast cancer EO771 cell line and human breast cancer MDA-MB-468 cell line were cultured in Dulbecco's Modified Eagle Medium (DMEM) supplemented with 10% fetal bovine serum (FBS) and 1% penicillin/streptomycin.

Primary breast tumor cell culture: Mouse and human breast tumors were cut into small pieces with diameter ˜2-4 mm, minced by the gentle MACS Dissociator (Miltenyi Biotec) and digested using the tumor isolation kits (Miltenyi Biotec, mouse, Cat. No. 130-096-730; human, Cat. No. 130-095-929). Detailed isolation procedure followed the manufacturer's instructions. Single cells from mouse tumor tissue were cultured with DMEM/F12 culture medium containing 10% FBS, 2 mM

Ultraglutamine I and 1% penicillin/streptomycin overnight. Single cells from patient samples were cultured in DMEM/F12 culture medium containing 10% FBS, 1% penicillin/streptomycin and 10 ng ml-1 human IL2.

Next, suspended cells were removed and the adherent cells were trypsinized and used to generate tumor organoids. Human breast cancer patient samples were provided by the Tissue Procurement and Distribution Core of Indiana University Simon Cancer Center (Table 2).

TABLE 2
Clinical information of human breast cancer samples.

| | Patient | | | | |
|---|---|---|---|---|---|
| Case | sample # | Sex | Age | Surgery | Diagnosis |
| 1 | EH1903-10 | F | 61 | mastectomy | Papillary carcinoma |
| 2 | EH1904-10 | F | 34 | mastectomy | Inflammatory |
| | | | | | breast cancer |
| 3 | EH1905-01 | F | 34 | mastectomy | Triple negative |
| | | | | | breast cancer |
| 4 | UHB1908-15 | F | 62 | mastectomy | Invasive ductal |
| | | | | | carcinoma (ER+) |
| 5 | UHB1907-03 | F | 68 | mastectomy | Invasive ductal |
| | | | | | carcinoma |
| | | | | | (ER/PR+, HER2−) |
| 6 | EHB1909-02 | F | 85 | mastectomy | Invasive ductal |
| | | | | | carcinoma (ER/PR+, |
| | | | | | HER2 equivical ) |
| 7 | EHB2009-01 | F | 48 | mastectomy | Invasive ductal |
| | | | | | carcinoma (TNBC) |
| 8 | UH2009-02 | F | 55 | mastectomy | Invasive ductal |
| | | | | | carcinoma (ER+, |
| | | | | | PR−, HER2−) |
| 9 | EH1904-07 | F | 65 | mastectomy | Invasive ductal |
| | | | | | carcinoma (ER+, |
| | | | | | PR+, HER2−) |
| 10 | UHB1908-09 | F | 59 | Partial | Recurrent invasive |
| | | | | mastectomy | ductal carcinoma |
| | | | | | (ER+/PR+) |

Breast tumor organoid culture: Single cells isolated from EO771-derived tumors in tumor-bearing C57BL/6 mice were suspended into cold mouse breast cancer culture medium containing 10% Matrigel (Corning) with a concentration of 2×105 cells ml⁻¹. The cells (2 ml) were seeded into each well of pre-warmed 6-well microplate with ultra-low attachment surface (Corning) and cultured at 37° C. with 5% CO2. The same volume of fresh culture medium was added every other day. After 7-day culture, tumor organoids were collected using cell strainers with pore size of 70 and 150 μm. For human tumor organoid culture, 2 ml of NY-ESO-1+ MDA-MB-468 cells with a concentration of 2×10⁵ cells ml-1 were seeded into each well of the prewarmed 6-well microplate with ultra-low attachment surface. The cells were cultured for two days to generate tumor spheroids. The resulting spheroids were mixed with cancer associated fibroblasts (CAFs) with a ratio of 2:1 (tumor cells versus CAFs) and cultured in the human breast cancer organoid culture medium3 for 5 days. Finally, the MDA-MB-468 tumor organoids with diameter of 70˜150 μm were used for co-culture with NY-ESO-1-specific human CD8+ T cells (ASTARTE Biologics). Patient-derived organoids (PDOs) were cultured following the same procedures as mouse EO771 tumor organoids using human breast cancer organoid culture medium.

Hypoxia test in breast tumor organoids: The Image-iT™ Green Hypoxia reagent (Invitrogen) was used to stain tumor organoids. The protocol follows the manufacturer's instructions. Fluorescent images were captured under a Leica DM4B microscope. The mean fluorescence intensity is the mean intensity of the projected area of hypoxia in tumor organoids, which was quantified using ImageJ software 1.50e.

Tumor cell viability assay: For 2D tumor cell culture, tumor cells were seeded into 96 well plates at 2,000 cells per well and cultured for 72 h. For tumor organoids, tumor organoids with diameter between 70 and 150 μm were seeded at 50 organoids per well into 96-well microplates with ultra-low attachment surface (Corning). Cells or tumor organoids were treated with indicated compound at the final concentration of 1 μM. After 3-day culture, tumor organoids were dissociated into single cells using TrypLE Express (Corning) at 37° C. with a shaking velocity of 500 rpm on a thermomixer (Eppendorf) for 15 min. The 96-well microplates were added with 10 μl/well of Premix WST-1 reagent (TaKaRa) and incubated for 1 h. The microplates were read at absorbance of 450 nm on the Epoch microreader (BioTek). The toxicity of the epigenetic drugs was quantified using the premix WST-1 reagent according to the manufacturer's protocol.

Cytotoxicity of CD8+ T cells in the co-culture with tumor organoids We generated mouse EO771 (Luc+OVA+) cell line that presents OVA257-264 antigen (from chicken ovalbumin) and human MDA-MB-468 (NY-ESO-1+) cell line that presents NY-ESO-1 antigen for use in the T cell-mediated cytotoxicity assay. OVA-specific CD8+ T cells were isolated from the spleen of OT-I mouse that contains transgenic inserts of Tcra-V2 and Tcrb-V5 genes. The transgenic T cell receptor can recognize the ovalbumin peptide SIINFEKL (OVA257-264; SEQ ID NO: 55). Anti-NY-ESO-1 human CD8⁺ T cells (CELLERO™) were used to recognize the NY-ESO-1+ MDA-MB-468 cells. Autologous human CD8+ T cells were isolated from patient tumor tissues and amplified in the DMEM/F12 medium supplemented with 10% FBS, 2 mM Ultraglutamine I, 1% penicillin/streptomycin and 10 ng mL-1 of human IL2 for 7 days. The CD8+ T cells were stimulated by the T-activator CD3/CD28 Dynabeads (Gibco, 11452D for mouse and 11131D for human) for 48 h prior to use for cytotoxicity assay. Mouse and human tumor organoids with diameter between 70 and 150 μm were collected and mixed with corresponding CD8+ T cells in DMEM/F12 medium supplemented with 10% FBS, 2 mM Ultraglutamine I, 1% penicillin/streptomycin and 10 ng mL-1 IL2 (Biolegend, mouse: 575402; human: 589102) at a ratio of organoids versus T cells at 1:200 in the microplates with ultra-low attachment surface. The cells were co-cultured for 24 h and subject to further analyses, such as optical and fluorescence imaging, luciferase assay or flow cytometry.

Epigenetic inhibitor screen on CD8+ T cell-mediated cytotoxicity: The epigenetic inhibitor library (Cayman chemical, Cat #11071, Batch #0522205, data not shown) was used for drug screen. Three different screen methods were conducted. In tumor organoid-T cell co-culture, EO771 (Luc+OVA+) tumor organoids with diameter between 70 and 150 μm were seeded with 100 organoids/well and treated with 1 μM of drug for 48 h. The tumor organoids were centrifuged at 100 rcf for 5 min. The medium were replaced with fresh T cell culture medium containing OVA-specific T cells. A total of 20,000 T cells were added in each well. After co-culture for 24 h, cytotoxicity of tumor cells was measured using a Dual-luciferase report 1000 assay system (Promega) in which luciferase expressed exclusively in the live EO771 (Luc+OVA+) cells was detected by a BioTek Cytation5 imaging reader. Low levels of luciferase from survived tumor cells refer to high cytotoxicity in the co-culture. The luciferase intensity in each well was subtracted by the background intensity for post-data analysis. In 2D tumor cell-T cell co-culture, EO771 (Luc+OVA+) tumor cells were seeded at a density of 4,000 cell/well and treated with drug for 48 h. The medium were replaced and the tumor cells were mixed with CD8+ T cells at a ratio of 1:5 in 96-well microplate (Corning) for overnight. The same protocol as above was used to measure the cytotoxicity of tumor cells. For OVA antigen presentation assay, EO771 (GFP+Luc+OVA+) tumor cells were treated with indicated drugs for 48 h, stained with APC-conjugated OVA peptide SIINFEKL (SEQ ID NO: 55) antibody for 15 min, and then were subjected to flow cytometry analysis. The mean fluorescence intensities (MFI) represents the OVA expression levels.

To evaluate intra-tumor infiltration of T cells, EO771 (Luc+OVA+) tumor organoids were treated with indicated drugs for 48 h, and then co-cultured with OT-I T cells. After co-culture for 48 h, the organoids with T cells inside or attached were dissociated by TrypLE Express (Thermo Fisher) into single cells and stained with APC/Cy7-conjugated anti-mouse CD8 (Biolegend) and SYTOX Blue reagent for 15 min. The CD8+ T cell proportions from tumor organoids were analyzed by flow cytometry.

T cell effector function analysis: We measured cytokine and cytolytic granule production (IFNγ, GZMB and TNFα) of CD8+ T cells from mouse tumor samples and tumor organoid-T cell co-culture. Briefly, CD8+ T cells were placed into the 24-well plate at 1×106 cells/well, stimulated with 1 μM ionomycin and 50 ng ml-1 phorbol 12-myristate 13-acetate (PMA) (Sigma-Aldrich) for 4 h, in the presence of 5 μg ml-1 Brefeldin A (BFA), with the purpose to amplify the expression of intracellular cytokines61. The cells were stained with APC/Cy7-conjugated anti-CD8a (Biolegend) for 15 min and then fixed by 4% PFA. After washing, cells were stained with PerCP/Cy7-conjugated anti-GZMB, APC-conjugated anti-IFNγ and PE-conjugated anti-TNFα (Biolegend) for 15 min. In flow cytometric analyses, T cells stained with isotype control antibodies were used as negative controls for gating the cytokine or granule-producing cells. For T cells from tumor organoid-T cell co-culture, tumor organoids were first digested into single cells with TrypLE Express at 37° C. before APC/Cy7-conjugated anti-CD8a staining. In the IFNγ and TNFα secretion assay (Biolegend), T cells were stimulated with ionomycin and PMA without the presence of BFA. The media were collected for ELISA assay.

Immunohistochemistry (IHC) and Immunofluorescence (IF) analysis of tumor organoids: Tumor organoids were gently centrifuged at 100 rcf from organoid culture medium and then embedded in 2% agar. Tumor organoids were fixed in 4% paraformaldehyde solution for overnight at room temperature. The samples were replaced with 70% ethanol and later embedded in paraffin blocks. Paraffin sections were cut at 5 μm thickness, attached on slides, heated at 60° C. for 1 h and then overnight at 37° C. The slides were stained with hematoxylin and eosin (H&E). IHC staining follows the protocol described62. Mouse-specific antibodies for CD8α (Cell Signaling Technology, clone D4W2Z, 1:400 dilution), cleaved caspase3 (Cell Signaling Technology, clone D175, 1:400 dilution) and Ki67 (Cell Signaling Technology, clone D3B5, 1:400 dilution) were used for IHC staining. The DAB peroxidase substrate kit (Vector laboratories Inc.) was used to visualize the antibodies. For IF analysis, the sample slides were blocked 3% BSA for 40 min after hydration. The slides were stained with primary antibodies including EpCAM (Abcam, 1:400 dilution), FITC-conjugated anti-α-SMA (Genetex, 1:400 dilution) and AF594-conjugated anti-CD31 (Biolegend) for 1 h. For EpCAM staining, the slide was further stained with secondary antibody Alexa Fluor 647-conjugated anti-rabbit IgG (Biolegend, 1:200) for 40 min. The fluorescence images were captured under a Leica DM4B microscope.

Immunoblotting: Immunoblotting assay was performed as described in the study63. Briefly, EO771 cells expressing lentiviral control shRNA or B2M shRNA was lysed in pre-cold RIPA buffer (50 mM Tris, pH 7.4, 150 mM NaCl, 1% nonidet P-40, 0.5% sodium deoxycholate, and 0.1% sodium dodecyl sulfate) supplemented with protease inhibitor cocktail (Roche) and phosphatase inhibitor mixture (Thermo Scientific). The protein concentration from cell lysates was determined using BCA kit (Thermo Fisher Scientific). 20 μg of cell lysate from each sample was used for polyacrylamide gel electrophoresis. The polyvinylidene difluoride (PVDF) membrane with the transferred proteins was blocked with 5% non-fat milk overnight, and then incubated with primary antibody B2M (Abcam) or beta-actin (Abgent) at room temperature for 2 h. The membrane was washed three times and incubated with HRP-conjugated anti-mouse or anti-rabbit secondary antibody at a concentration of 1:5,000 at room temperature for 1.5 h. The membrane was washed three times, incubated with chemiluminescent substrates (western lightning plus ECL kit, PerkinElmer), and exposed to X-ray film for imaging analysis.

Animal studies: All animal experiments were performed in accordance with the National Institutes of Health's Animal Use Guidelines and protocols approved by the Animal Care and Use Committee of Indiana University School of Medicine. To examine the tumorigenic potential of tumor organoids, tumor organoids with different sizes (diameter of 30-70, 70-150, and 150-500 μm) were orthotopically injected into fat pad of the 4th mammary gland in female C57BL/6 mice. Each mouse was injected with a total of 2×105 tumor cells. 5 mice were used in each group. Tumors were measured in two dimensions using a manual caliper. Tumor volume was calculated using the following formula: V=0.5×length×width×width. Tumor volume was measured every 3 days. Tumors were harvested 22 days after injection. The tumor growth, volume and weight were analyzed. To evaluate the anti-tumor effect of drugs in immunocompetent C57BL/6 mice and immunodeficient nude (NU/J) mice, 2×105 EO771 cells were orthotopically injected into fat pad of the 4th mammary gland in female mice. Once tumors were established at day 10, the tumor-bearing mice were randomly divided into each treatment group (n=8 mice/group), and were treated with vehicle control, BML-210, CUDC-101, GSK-LSD1, PFI-1 or Bromosporine (20 mg kg-1 by intraperitoneal injection) 3 times/week for 2 weeks. For CD4/CD8 depletion experiment, the tumor-bearing mice were randomly divided into 7 groups (n=8 mice/group) including isotype control (rat IgG2a, Bioxcell), isotype control+CUDC-101, isotype control+BML-210, anti-CD8 (Clone: 53-6.72, Bioxcell)+CUDC-101, anti-CD8+BML-210, anti-CD4 (Clone: GK1.5, Bioxcell)+CUDC-101, or anti-CD4+BML-210. Intraperitoneal injection of inhibitors (20 mg kg-1) and anti-CD4/CD8 (10 mg kg-1) were administrated to the mice by 3 times/week for 2 weeks. The tumors were harvested at day 18 (NU/J mice) or day 28 (C57BL/6 mice) for further analyses. In immune checkpoint blockade experiment, 2×105 EO771 cells were orthotopically injected into fat pad of the 4th mammary gland in female C57BL/6 mice. From day 10, the tumor-bearing mice were randomly divided into each treatment group (n=8 mice/group), and the mice were treated with IgG2a isotype control antibody (clone 2A3, Bioxcell), BML-210 (20 mg kg-1), anti-PD-1 (200 μg/mouse, Bioxcell), or BML-210 and anti-PD-1 combo. The treatment was done 3 times/week for 2 weeks. Tumors were harvested at day 26 post injection and the tumors were analyzed by IHC staining, flow cytometry, ELISA or CyTOF assay.

Flow Cytometry: Sample acquisition was done using LSR Fortessa X-20 or LSR Fortessa (BD Biosciences) and data were analyzed using FlowJo v10.6.0 software. Live/dead cells were assessed using SYTOX Blue (Invitrogen, Dilution 1:100). For cell staining involving cell permeabilization, the BD cytofix/cytoperm fixation permeabilization kit (BD Biosciences) was used. The PBS buffer containing 0.5% BSA and 10% FBS were used to dilute antibodies. The antibodies used in flow cytometrical analyses include: AF594-conjugated anti-mouse CD31 (Biolegend, Dilution 1:100), APC-conjugated anti-mouse CD140a (Biolegend, Dilution 1:400), PE-conjugated anti-mouse CD326 (Biolegend, Dilution 1:100), AF647-conjugated anti-mouse CD326 (Biolegend, Dilution 1:100), BV405-conjugated anti-mouse CD45 (Biolegend, Dilution 1:100), PE/Cy7-conjugated anti-mouse CD3 (Biolegend, Dilution 1:100), AF700-conjugated anti-mouse CD4 (Biolegend, Dilution 1:100), APC/Cy7-conjugated anti-mouse CD8a (Biolegend, Dilution 1:100), APC-conjugated anti-mouse CD8 (Biolegend, Dilution 1:100), PE-conjugated anti-mouse CD11b (Biolegend, Dilution 1:200), AF647-conjugated anti-mouse CD11c (Biolegend, Dilution 1:200), PerCP/Cy5.5-conjugated anti-mouse Fn/80 (Biolegend, Dilution 1:100), BV421-conjugated anti-mouse I-A/I-E (Biolegend, Dilution 1:1000), BV650-conjugated anti-mouse CD19 (Biolegend, Dilution 1:100), APC-conjugated anti-mouse H-2Kb bound to SIINFEKL (SEQ ID NO: 55) antibody (Biolegend, Dilution 1:20), PerCP/Cy5.5 anti-mouse H-2Kb Antibody (Biolegend, Dilution 1:100), PE-Anti-Human HLA-A,B,C antibody (Biolegend, Dilution 1:100), FITC-conjugated anti-mouse IgG (Biolegend, Dilution 1:100), FITC-conjugated anti-Rat IgG (Biolegend, Dilution 1:100), APC-conjugated anti-mouse IFNγ (Biolegend, Dilution 1:100), PE-conjugated anti-mouse TNFα (Biolegend, Dilution 1:100), PerCP/Cyanine5.5-conjugated anti-human/mouse Granzyme B (Biolegend, Dilution 1:100), APC-conjugated anti-human IFNγ (Biolegend, Dilution 1:100), PE-conjugated anti-human TNFα (Biolegend, Dilution 1:100). The compensation was performed using CompBeads for negative control (BD Biosciences), anti-mouse Ig,κ (BD Biosciences), anti-rat Ig,κ and anti-hamster Ig,κ (BD Biosciences). The beads were stained with the corresponding antibodies separately under the same conditions as the cells were stained in each experiment. SYTOX Blue reagent was used to stain dead cells. Half of the live cells were incubated at 60° C. for 2 min to enable membrane permeable and then mixed with the other half incubated at 37° C. The mixed cells were stained with SYTOX Blue for 15 mins and used for compensation. For all the data analysis, doublet exclusion was performed and only single cells were analyzed. Dead cell populations were also excluded from analysis if the experiment were not involved in cell killing. One million events per sample were collected for immune profiling analysis in the animal experiments. For all in vitro experiments, at least ten thousand events per sample were collected for flow cytometry analysis.

Confocal Microscopy: OVA+ EO771 and NY-ESO-1+ MDA-MB-468 cells were seeded at 5×103 cell/well in the 8-well slide (Millicell EZ slide, R8AA09916) and treated with 1 μM drug (GSK-LSD1, CUDC-101 or BML-210) for 48 h. FITC-conjugated anti-mouse H-2Kb antibody (Biolegend, dilution 1:50) and FITC-conjugated anti-HLA-A2 (Biolegend, dilution 1:50) were used to stain OVA+ EO771 and NY-ESO-1+ MDA-MB-468 cells, respectively, for 2 h. For cell nucleus staining, cells were incubated with 0.5% Triton X-100/PBS for 10 min and stained with DAPI (Sigma-Aldrich, dilution 1:100) for 15 min. The slides were rinsed in PBS twice and mounted with ProLong Gold anti-fade mountant (Invitrogen, P36930). Fluorescence images were captured using Leica TCS SP8 (upright high speed multiphoton) confocal imaging system. 3-D images were analyzed by the Imaris x64 8.1.2 software.

Mass Cytometry (CyTOF): EO771 tumors in C57BL/6 mice were harvested and dissociated into single cells. 1×106 single cells from each sample were suspended in ice-cold buffer (PBS with 0.5% BSA and 0.02% Azide). The antibody panel (Table 1) was used for sample staining. Samples were analyzed using the CyTOF 2 Mass Cytometer (Fluidigm). All the samples were done on bead-based normalization before analysis on Cytobank. CyTOF data were analyzed using viSNE analysis64 in the Cytobank platform. The viSNE analysis uses the Barnes-Hut implementation of the t-Distributed Stochastic Neighbor embedding (tSNE) algorithm. The cell populations were visualized on viSNE as an overlay plot.

Quantitative reverse transcription PCR (RT-PCR): Mouse EO771 cells were treated with and without BML-210 drug for 48 h. Total RNA was isolated from the cells using TRIzol (Ambion, Lot: 260808) and the miRNeasy Mini kit (Qiagen, 157029493, Thermo Scientific). The quality of the RNA was quantified by a Nano Drop 2000 spectrophotometer. A total of 1 μg RNA was reverse transcribed into cDNA using the qscriptXLT cDNA superMix reagent (Quantabio, 66141329). Quantitative PCR was performed using SYBR Green fastmix low Rox reagent (TaKaRa) on a Quantstudio real-time PCR system (ThermoFisher Scientific). The PCR primer sequences are listed in Table 3.

TABLE 3
RT-qPCR Primers

| Mouse primers | | | Human primers | | |
|---|---|---|---|---|---|
| Primer | Sequence | SEQ | Primer | Sequence | SEQ |
| Name | 5′ to 3′ | ID No. | Name | 5′ to 3′ | ID No. |
| mB2M qF | TTCTGGTGCTT | 1 | hB2M qF | GAGGCTATCCA | 29 |
| | GTCTCACTGA | | | GCGTACTCCA | |
| mB2M qR | CAGTATGTTCG | 2 | hB2M qR | CGGCAGGCATA | 30 |
| | GCTTCCCATTC | | | CTCATCTTTT | |
| mCALR | TGGCTGCTCCC | 3 | hCALR | CCTGCCGTCTAC | 31 |
| qF | AATAATGTCT | | qF | TTCAAGGAG | |
| mCALR | GAGGGTAGTG | 4 | hCALR | GAACTTGCCGG | 32 |
| qR | ACCAAAAGAT | | qR | AACTGAGAAC | |
| | GG | | | | |
| mITGAV | CCGTGGACTTC | 5 | hITGAV | ATCTGTGAGGT | 33 |
| qF | TTCGAGCC | | qF | CGAAACAGGA | |
| mITGAV | CTGTTGAATCA | 6 | hITGAV | TGGAGCATACT | 34 |
| qR | AACTCAATGG | | qR | CAACAGTCTTT | |
| | GC | | | G | |
| mSEC22B | CTGACGATGAT | 7 | hCTSV | CGTGACGCCAG | 35 |
| qF | CGCCCGTG | | qF | TGAAGAATCA | |
| mSEC22B | TGCTTAGCCTG | 8 | hCTSV | CGCTCAGTGAG | 36 |
| qR | ACTCTGATACT | | qR | ACAAGTTTCC | |
| | G | | | | |
| mSEC23A | AGATGGGGTC | 9 | hSEC22B | CCGGGACCTTC | 37 |
| qF | CGGTTCAGTT | | qF | AACAATATCAG | |
| mSEC23A | GGTAGGTCGG | 10 | hSEC22B | GCAAAAGCCAA | 38 |
| qR | GTCTCTCCTT | | qR | CTTCTTAGGGA | |
| mSEC24A | TCCTGTCCACA | 11 | hSEC23A | GGAGTCCGATT | 39 |
| qF | ATACACTGATG | | qF | TAGTTGGAATG | |
| | T | | | T | |
| mSEC24A | GAACCACCGT | 12 | hSEC23A | AGGTCTCTCTTT | 40 |
| qR | AGTTCGACTGT | | qR | CAGTGGTGT | |
| mSEC24C | CTGGCCGGAAT | 13 | hSEC24A | ATGTCCCAGCC | 41 |
| qF | GCAGATCAG | | qF | GGGAATAC | |
| mSEC24C | TGAGGATAGG | 14 | hSEC24A | AGGACCGTTGG | 42 |
| qR | AGCCGTATGG | | qR | TGTAGGGAG | |
| | A | | | | |
| mSEC24D | GGAGAGGTCTT | 15 | hSEC24C | AACGTCAACCA | 43 |
| qF | TGTTCCTTTGT | | qF | GTCAGTTCCA | |
| | T | | | | |
| mSEC24D | GTCTCTGTTCT | 16 | hSEC24C | GGCTCCATAGG | 44 |
| qR | TGAGCTTCCC | | qR | GAATGGCG | |
| mPSMB4 | ATGGAAGCGTT | 17 | hSEC24D | GTCAACAAGGT | 45 |
| qF | TTGGGAGTCA | | qF | TACGTGGCTA | |
| mPSMB4 | GTTCTGGGTCC | 18 | hSEC24D | TAGTGCCCATA | 46 |
| qR | GAGTGATGG | | qR | ATGAGGTGGA | |
| mPSMD2 | TGGACGAGAC | 19 | hPSMD8 | GCCGTAAATCA | 47 |
| qF | AAGACACCC | | qF | GGCGGTCT | |
| mPSMD2 | CTCCACGAGCA | 20 | hPSMD8 | GCCCTTGAGTT | 48 |
| qR | TCTCCAGTTC | | qR | GCTCGTACA | |
| mPSMD8 | GGCATGTACG | 21 | hPSMD11 | GCCTCCATCGA | 49 |
| qF | AGCAACTCAA | | qF | CATCCTCC | |
| | G | | | | |
| mPSMD8 | GCTCAAGCAG | 22 | hPSMD11 | GAGCTGCTTTA | 50 |
| qR | AACCAACTTCA | | qR | GCCTTGCTG | |
| mPSME4 | AGCGTCAACA | 23 | hPSMB4 | GAAGCGTTTTT | 51 |
| qF | AGATAAGAAT | | qF | GGGGTCGC | |
| | GCT | | | | |
| mPSME4 | GCCCGATTCCT | 24 | hPSMB4 | GAGTGGACGGA | 52 |
| qR | ATATGCTCAAA | | qR | ATGCGGTA | |
| mPSMD11 | GCAGGAGGTC | 25 | HLA-A | AAAAGGAGGGA | 53 |
| qF | GAGCTATGTTT | | qF | GTTACACTCAG | |
| | | | | G | |
| mPSMD11 | TGAGAACCCA | 26 | HLA-A | GCTGTGAGGGA | 54 |
| qR | AATGCAATGCT | | qR | CACATCAGAG | |
| | T | | | | |
| H-2K1 | ACCAGCAGTA | 27 | | | |
| qF | CGCCTACGA | | | | |
| H-2K1 | AACCAGAACA | 28 | | | |
| qR | GCAACGGTCG | | | | |

RNA sequencing: Total RNA was extracted from EO771 cells using TRIzol (Ambion) and the miRNeasy Mini kit (Qiagen). The quantity and quality of the RNA was evaluated by an Agilent Bioanalyzer 2100. 100 ng of total RNA from each sample was used, from which ribosomal RNA was removed by the QIAseq FastSelect rRNA Removal HMR Kit (Qiagen). The cDNA library was constructed using the KAPA RNA Hyper Prep Kit (Roche Corporate) and the quality was accessed by the Qubit and Agilent Bioanalyzer. The library pool was sequenced in 100b paired-end read format on NovaSeq 6000 (Illumina) at the Center for Medical Genomics, Indiana University School of Medicine (IUSM).

RNA-seq analysis: The reads were mapped to the mouse genome mm10 using STAR (v2.5)65. RNA-seq aligner with the following parameter: “--outSAMmapqUnique 60”. Uniquely mapped sequencing reads were assigned to GENCODE M22 genes using feature Counts (v1.6.2)66 with the following parameters: “-s 2 -p -Q 10”. The count data were filtered using read count per million (CPM) >10 in more than two of the samples, normalized using TMM (trimmed mean of M values) method and subjected to differential expression analysis using edgeR package (v3.28.1) 67,68 in R software (version 3.5.3). A false discovery rate (FDR) cut-off of 0.01 was used to determine significantly differential expressed genes (DEGs). The data were visualized with volcano plot using MATLAB. The gene enrichment analysis on DEGs from the Kyoto Encyclopedia of Genes and Genomes (KEGG) pathway database and Gene Ontology database was performed using the Database for Annotation, Visualization and Integrated Discovery (DAVID) functional analysis69. The analysis was visualized using MATLAB with q value (−log 10) and gene number in each GO and pathway. Gene set enrichment analysis (GSEA) of the antigen processing and presentation pathway was carried out with GSEA software70. The significantly differential genes from GSEA were plotted with heatmap by using the Complex Heatmap package71 in R software (version 3.5.3).

Statistical analysis: Data were analyzed using GraphPad Prism 8. Fluorescence intensity from images was quantified using ImageJ software (Version 1.50e). Flow cytometry data were analyzed using FlowJo version 10.6.0. Group sizes and error bars are indicated as needed. Statistical analysis of data sets higher than two groups with one independent variable was performed using one-way ANOVA. Statistical analysis of multiple groups of two factors was performed using two-way ANOVA. All the data deviations are indicated as mean±SEM. p value indicated as * (p<0.05), ** (p<0.01), *** (p<0.001) and **** (p<0.0001). p<0.05 is considered significantly different.

TABLE 4
Reagents and Resources.

| | | |
|---|---|---|
| REAGENT or RESOURCE | SOURCE | CATALOG |

Antibodies

| | | |
|---|---|---|
| iFluor488-conjugated Phalloidin | Abcam | ab176753 |
| Rbmab-EPCAM antibody | Abcam | ab32392 |
| anti-mouse PD-1 | Bioxcell | BE0146; Clone: RMP1-14 |
| Rat IgG2a isotype control | Bioxcell | BE0089 |
| Anti-CD4 | Bioxcell | BE0003-1; Clone: GK1.5 |
| Anti-CD8 | Bioxcell | BE0004-1; Clone: 53-6.72 |
| beta-2 microglobulin antibody | Abcam | Ab175031 |
| Beta-Actin antibody | Abgent | AM1829b |
| anti-mouse CD8α (D4W2Z) antibody, rabbit monoclonal | Cell Signaling Technology | 98941S |
| Cleaved caspase 3 (Asp175) antibody | Cell signaling Technology | 9661S |
| mouse Ki-67 antibody | Cell signaling Technology | 12202S |
| AF594-conjugated anti-mouse CD31 | Biolegend | 102520 |
| APC-conjugated anti-mouse CD140a | Biolegend | 135907 |
| PE-conjugated anti-mouse CD326 | Biolegend | 118206 |
| AF647-conjugated anti-mouse CD326 | Biolegend | 118212 |
| FITC-conjugated Mouse α-Smooth Muscle Actin | GeneTex | GTX72531 |
| AF647-conjugated Donkey anti-rabbit IgG | Biolegend | 406414 |
| APC/Cy7-conjugated anti-human CD8a | Biolegend | 300926 |
| APC-conjugated anti-human IFNγ | Biolegend | 502512 |
| PE-conjugated anti-human TNFα | Biolegend | 502909 |
| APC-conjugated anti-mouse IFNγ | Biolegend | 505810 |
| PE-conjugated anti-mouse TNFα | Biolegend | 506306 |
| PerCP/Cyanine5.5-conjugated anti-human/mouse Granzyme B | Biolegend | 372212 |
| BV605-conjugated anti-mouse CD45 | Biolegend | 103139 |
| PE/Cy7-conjugated anti-mouse CD3 | Biolegend | 100220 |
| AF700-conjugated anti-mouse CD4 | Biolegend | 100430 |
| APC/Cy7-conjugated anti-mouse CD8 | Biolegend | 100714 |
| APC-conjugated anti-mouse CD8 | Biolegend | 126613 |
| PE-conjugated anti-mouse CD11b | Biolegend | 101208 |
| AF647-conjugated anti-mouse CD11c | Biolegend | 117312 |
| PerCP/Cy5.5-conjugated anti-mouse Fn/80 | Biolegend | 123128 |
| BV421-conjugated anti-mouse I-A/I-E | Biolegend | 107631 |
| BV650-conjugated anti-mouse CD19 | Biolegend | 115541 |
| PE-conjugated anti-mouse H-2Kb bound to SIINFEKL | Biolegend | 141603 |
| (SEQ ID NO: 55) antibody | | |
| APC-conjugated anti-mouse H-2Kb bound to SIINFEKL | Biolegend | 141606 |
| (SEQ ID NO: 55) antibody | | |
| FITC-Anti-Human HLA-A, B, C antibody | Biolegend | 311403 |
| FITC-Anti-Human HLA-A2 antibody | Biolegend | 343303 |
| FITC-conjugated anti-H-2Kb | Biolegend | 116505 |
| FITC-conjugated anti-Rat IgG | Biolegend | 407505 |
| FITC-conjugated anti-mouse IgG | Biolegend | 406001 |
| SYTOX ™ Blue dead cells | Invitrogen | 2078615 |

Chemicals, Peptides, Proteins and Consumables

| | | |
|---|---|---|
| Matrigel | Corning | 356255 |
| Mouse IL2 | Biolegend | 575402 |
| Human IL2 | Biolegend | 589102 |
| Image-iT ™ Green Hypoxia reagent | Invitrogen | I14833 |
| phorbol 12-myristate 13-acetate | Sigma-Aldrich | 16561-29-8 |
| Ionomycin | Sigma-Aldrich | 56092-82-1 |
| Brefeldin A | Biolegend | 420601 |
| Cell strainers (40 μm) | Fisher Scientific | 07201430 |
| Cell strainers (70 μm) | Fisher Scientific | 07201431 |
| Cell strainers (100 μm) | Fisher Scientific | 07201432 |
| Cell strainers (150 μm) | pluriStrainer | 43-50150-03 |
| Cell strainers (300 μm) | pluriStrainer | 43-50300-03 |
| Cell strainers (500 μm) | pluriStrainer | 43-50500-03 |
| Mouse tumor dissociation kit | Milenyi Biotec | 130-096-730 |
| Human tumor dissociation kit | Milenyi Biotec | 130-095-929 |
| Human CD8+ magnetic beads | Milenyi Biotec | 130-045-201 |
| Mouse CD8+ magnetic beads | Milenyi Biotec | 130-117-044 |
| Dynabeads Human T activator CD3/CD28 | Gibco | 11131D |
| Dynabeads Mouse T activator CD3/CD28 | Gibco | 11452D |
| BD comp Beads Negative Control | BD Biosciences | 51-90-9001291 |
| BD comp Beads Anti-Mouse Ig, κ | BD Biosciences | 51-90-9001229 |
| BD comp Beads Anti-Rat and Anti-Hamster Ig, κ | BD Biosciences | 51-90-9000949 |
| low-attachment surface 96-well microplate | Corning | 3474 |
| low-attachment surface 24-well microplate | Corning | 3473 |
| low-attachment surface 6-well microplate | Corning | 3471 |
| 8-Well glass EZ Slide | Millicell | R8AA09916 |
| RNeasy mini-isolation kit | QIAGEN | 157029493 |
| QIAseq FastSelect rRNA Removal HMR Kit | QIAGEN | 334387 |
| KAPA RNA Hyper Prep Kit | Roche Corporate | KK8541 |
| QscriptXLT cDNA super Mix | Quantabio | 66141329 |
| SYBR Green fastmix low Rox | TaKaRa | 4385610 |
| Lipofectamine 3000 reagent | Thermo Fisher Scientific | 25530049 |
| Premix WST-1 assay | TaKaRa | MK400 |
| Prolong Gold anti-fade medium | Invitrogen | P36930 |
| Cytofix/cytoperm fixation/permeabilization kit | BD Biosciences | 554714 |
| TRIzol | Ambion | 260808 |
| RBC lysis buffer(10X) | Biolegend | 420301 |
| Dual-luciferase report 1000 assay system | Promega | E1980 |
| ELISA MAX ™ Deluxe Set Human TNF-α | Biolegend | 430204 |
| ELISA MAX ™ Deluxe Set Mouse TNF-α | Biolegend | 430904 |
| ELISA MAX ™ Deluxe Set Human IFN-γ | Biolegend | 430104 |
| ELISA MAX ™ Deluxe Set Mouse IFN-γ | Biolegend | 430804 |
| Epigenetic drug library | Cayman Chemical | 11071 |

Cells and Cell Lines

| | | |
|---|---|---|
| MDA-MB-468 | ATCC | Cat# HTB-132; RRID: CVCL_0419 |
| HEK293T | ATCC | Cat# CRL-3216; RRID: CVCL_0063 |
| EO771 | CH3 BioSystems | Cat# 940001; RRID: CVCL_GR23 |
| Anti-NY-ESO-1 human CD8+ T cells | ASTARTE Biologics | 1093-4245MA19 |
| Mouse beta-2-microglobulin (B2M) shRNA bacterial | Sigma-Aldrich | SHCLNG-NM_00935, |
| Glycerol stock | | TRCN0000288438 |

Software and Algorithms

| | | |
|---|---|---|
| FlowJo | N/A | FlowJo version 10.6.0 |
| GraphPad | N/A | GraphPad Prism 8.0 software |
| Matlab | MathWorks Inc. | Version 8.2.1, R2016b |
| Mass Cytometry | Cytobank | https://cytobank.org/ |
| R | N/A | https://www.R-project.org/ |
| Gene Set Enrichment Analysis (GSEA) | N/A | http://software.broadinstitute.org/gsea/ |

### Example 2

### Generation and Characterization of Breast Tumor Organoids

To facilitate our high-throughput drug screen for breast cancer immunotherapy, we established a drug screen system that is composed of OVA257-264 antigen-presented mouse breast cancer EO771 cell-derived tumor organoids and CD8+ T cells isolated from OT-I transgenic mice that recognize the OVA antigen in the context of H2Kb. GFP+Luc+OVA+ EO771 cells were generated, in which green fluorescence protein (GFP) is used to label tumor cells and luciferase is expressed for measuring T cell-mediated cytotoxicity in the screen. The engineered EO771 cells were orthotopically injected into the fat pad of 4th mammary gland in female C57BL/6 mice and the resulting tumors were harvested to generate tumor organoids as described in the Methods. To optimize tumor organoids for a high-throughput drug screen, we studied the size, heterogeneity, hypoxia, composition and culture time of the tumor organoids. Organoid size and heterogeneity with increasing cell seeding concentrations were determined. The organoid size variation was much greater as the cell seeding concentration increased, especially when higher than 2×10⁵ cells/ml (2 ml per well) in the 6-well microplate. Organoid sizes were concentrated around 70 to 150 μm in diameter at the seeding concentration of 2×105 cells/ml after 7-day culture. We started to observe significantly high cytotoxicity of CD8+ T cells from 12 h after co-culture with tumor organoids and increased up to 48 h. A moderate killing effect of T cells was desired for drug screen, as such the greater or weaker effect upon drug treatment could be detectable. The proportion of dead CD8+ T cells in the tumor organoids (70-150 μm) was ˜11% one day after co-culture, similar to what was observed in the 2D co-culture. However, the T cell death increased significantly at two days after co-culture with the tumor organoids, suggesting that the co-culture time is optimal around one day for measuring T cell activity. Hypoxia in tumor organoids became more severe as the organoid size increased. When the organoid size was between 100 and 150 μm in diameter, the oxygen levels at the core area of the organoids were ˜1-5%, which is similar to the hypoxia state observed in solid tumors. Tumor cell viability in the organoids markedly declined with increasing organoid size, particularly when the organoids were larger than 150 μm in diameter, indicating that hypoxia may cause non-specific tumor cell death if tumor organoids grow too large. Tumor organoids with modest cell death, physiologically relevant hypoxia, and relatively uniform size are optimal for a standardized high-throughput drug screen. With these measurements, we selected the tumor organoids with size of lower than 150 μm in diameter.

### Example 3

Tumor cells and their functional interaction with associated stroma in the tumor microenvironment impacts disease initiation and progression. To further validate the optimal size of tumor organoids, we assessed the tumorigenic potential of tumor organoids with different sizes in mouse syngeneic tumor models, where the EO771-derived breast tumor organoids with same tumor cell number were transplanted into C57BL/6 mice by orthotopic injection. Tumor organoids with diameter around 70-150 μm exhibited the highest level of tumorigenesis in terms of tumor volume and weight, in comparison with the larger or smaller organoids. The cellular composition of tumor organoids at different time points revealed that tumor cells (EpCAM+) grew faster than other cell types such as tumor associated fibroblast (CD140+) and vascular endothelial cells (CD31+). However, the cellular composition at day 7 was close to that of dissociated cells from the original tumor (day 1). In particular, the EO771 tumor organoids with the diameter of 70 to 150 μm after 7-day culture had 70.6% of EpCAM+ cells, 19.8% of fibroblast cells, and 6.7% endothelial cells, respectively. These cell types were also observed in immunofluorescence staining images of tumor organoids, representing the cellular composition determined by flow cytometry analysis. The results concluded that tumor organoids with size between 70-150 μm after 7-day culture well resemble their original tumors and retain strong tumorigenicity. Notably, CD45+ immune cells were excluded from the tumor organoids. Without a variety of pre-existing immune cells in tumor organoids, we were able to measure the tumor-specific T cell-mediated cytotoxicity in the co-culture of tumor organoids with the CD8+ T cells.

### Example 4

Screen of Epigenetic Inhibitors that Enhance T Cell Activity

Epigenetic modulation governs transcriptional program in tumor cells, which may impact their antigen presentation and response to the T cell-mediated effects. To screen small-molecule compounds that enhance the T cell-mediated cytotoxicity on tumor cells, a library of 141 compounds that modulate the activity of methyltransferases, demethylases, histone acetyltransferases (HATs), histone deacetylases (HDACs), and acetylated lysine reader proteins was applied (at the concentration of 1 μM) in the optimized 3D tumor organoid-T cell co-culture system. As comparison, we also used 2D tumor cell-T cell co-culture screen system in parallel. Tumor cell viability was measured as an indicator for the T cell-mediated cytotoxicity. Additionally, we also assessed the OVA antigen presentation of tumor cells in the drug-treated tumor organoids. OVA+Luc+EO771-derived tumor organoids with diameter between 70-150 μm were generated and used in the screen. To exclude the compounds with cytotoxic activity similar to that of chemotherapeutic drugs, we first assessed their cytotoxicity on tumor organoids without T cells co-cultured. Only those compounds (121 in total) without significant cytotoxicity at the concentration of 1 μM (P>0.05, in FIG. 1A) were selected for the immunological cytotoxicity screen in the next step, among which 8 compounds from the 3D co-culture system (FIG. 1B) and 15 compounds from the 2D co-culture system (FIG. 1C) exhibited significant activity in enhancing the CD8+ T cell-mediated cytotoxicity (P<0.05). One of the primary mechanisms for immune evasion of tumor cells is reduced antigen presentation, which prevents tumor cells from the recognition and destruction of cytotoxic T cells. A total of 25 compounds markedly promoted the OVA antigen presentation on the OVA+ EO771 cells (P<0.05, in FIG. 1D) in the 2D tumor cell culture. As expected, a reduced number of compounds were identified from the 3D tumor organoid screen than from the 2D tumor cell screen, and vice versa some compounds effective in the 3D system showed no activity in the 2D system, suggesting that tumor microenvironment and tumor stroma play an important role in modulating the immune response of tumor cells. T cell infiltration was evaluated in tumor organoids treated with the positive compounds screened from the 2D and organoid system. The compounds screened from the organoid system induce relatively high CD8+ T cell infiltration in the tumor organoids. By contrast, only a few of the compounds from 2D system induced T cell infiltration. Animal experiments confirmed that the compounds (PFI-1 and bromosporine) with poor T cell infiltration showed no anti-tumor effect. The results suggested that the organoid platform has the advantage over the 2D culture in identifying drugs for immune drug discovery. By combining the results from the 2D cell co-culture screen, tumor organoid co-culture screen, and antigen presentation screen, three compounds (CUDC-101, GSK-LSD1 and BML-210 were identified as drug candidates that increase the antigenicity of tumor cells and enhance their response to cytotoxic T cells.

### Example 5

In the tumor organoid and T cell co-culture system, treatment with CUDC-101, GSK-LSD1 or BML-210 drugs resulted in greater organoid dissociation and higher tumor cell death rates (Fig.), while the same treatments had no notable effect on the tumor organoids in the absence of T cells. Cytokine interferon gamma (IFNγ), cytolytic granule enzyme Granzyme B (GZMB), and tumor necrosis factor (TNFα) are the key indicators for cytotoxic T cell-mediated cytotoxicity. Their expression levels in the co-cultured CD8+ T cells were remarkably up-regulated when co-cultured with the drug-treated tumor organoids. Enzyme-linked immunosorbent assay (ELISA) also showed that these CD8+ T cells secreted much higher levels of IFNγ and TNFα in comparison with those T cells co-cultured with control untreated tumor organoids.

### Example 6

To validate the activity of these compounds in human breast tumor organoids derived from MDA-MB-468 cancer cells and human breast cancer-associated fibroblasts (CAFs), the human cancer antigen NY-ESO-1 was transduced into MDA-MB-468 cells, which can be recognized by mature human T cells specific for NY-ESO-1 bound to HLA-A2. Similar to the results from the mouse tumor organoid system, all the three compounds pronouncedly induced the T cell-mediated cytotoxicity (FIG. 2C). GZMB, IFNγ and TNFα expression levels in the CD8+ T cells from the treatment groups were also much higher than those in the CD8+ T cells from the control groups (FIG. 2D). Collectively, we identified three epigenetic modulator compounds, CUDC-101, GSK-LSD1 and BML-210, that promote cytotoxic T cell-dependent anti-tumor response.

### Example 7

### BML-210, CUDC-101 and GSK-LSD1 Exhibit Anti-Tumor Activity In Vivo.

Among the three compounds identified from our screen, GSK-LSD1 was reported to inhibit the histone demethylase LSD1 and promote CD8+ T cell infiltration and anti-tumor response in a mouse melanoma model, partly supporting the effectiveness of our tumor organoid screen system for immune drug discovery. No previous studies have been reported on the activity of BML-210 and CUDC-101 in cancer immunotherapy.

### Example 8

We first attempted to test whether the treatment of BML-210, CUDC-101 inhibits breast tumor growth in the context of immunocompetent animals. Mouse breast cancer EO771 cells were orthotopically implanted into the fat pad of mammary glands of female C57BL/6 mice. Once tumors were established (75-100 mm³), the mice were treated with BML-210 and CUDC-101 three times per week for two weeks (FIG. 3A). The tumor growth and weight in the BML-210 and CUDC-101 treatment groups were significantly suppressed in comparison with the control group (FIGS. 3B and 3C). Immune profile analysis revealed a higher tumor infiltration of total T cells (CD3+), CD4+ and CD8+ T cells in the tumors treated with each of the three drugs (FIG. 3D). The expression of GZMB, IFNγ and TNFα in the tumor-infiltrated CD8+ T cells was also up-regulated in the drug treatment groups (FIG. 3E), indicating enhanced cytotoxicity of the CD8+ T cells. Consequently, the tumor cell proliferation (Ki67+) from the drug treatment groups were markedly lower than the control group, while the numbers of apoptotic tumor cells (cleaved Caspase 3+) were increased in the drug treatment groups (FIGS. 3F and 3G), further suggesting the activity of the three drugs in tumor growth inhibition. Furthermore, the CD8+ T cells in the drug-treated tumors secreted more IFNγ and TNFα upon stimulation, a feature for the activity of CD8+ T cells. No visible damages were observed in the heart, kidney, spleen, liver and lung of mice treated with the two new drugs BML-210 and CUDC-101, suggesting the drug tolerability and low toxicity. To prove that the tumor growth inhibition was due to anti-tumor immune response instead of direct drug treatment effect on tumor cells, similar experiments were conducted with BML-210 and CUDC-101 in the immune-deficient nude (Nu/J) mice. No significant differences were observed on tumor growth and weight between the drug treatment groups and the control group. Depletion of CD8+ T cells from the drug-treated mice abolished the anti-tumor activity of CUDC-101 and BML-210, while depleting CD4+ T cells did not have much effect, suggesting that CUDC-101 and BML-210 suppressed tumor growth via CD8+ T cell-mediated cytotoxicity (FIGS. 3H and 3I).

### Example 9

Next, we wanted to confirm whether GSK-LSD1 promotes the immune response of breast tumors although its activity was reported in mouse melanoma models. In vivo studies of GSK-LSD1 were conducted in a way as described above for CUDC-101 and BML-210. As anticipated, the growth and weight of EO771 tumors in the GSK-LSD1 treatment group were markedly suppressed compared to the tumors in the control group. Immune analyses showed a higher tumor infiltration of T cells and enhanced cytotoxicity of the CD8+ T cells in the tumors treated with GSK-LSD1. Consistently, the tumor cell proliferation from the drug treatment group were much lower and the numbers of apoptotic tumor cells were increased in the GSK-LSD1 treatment group, supporting the activity of GSK-LSD1 in breast tumor growth inhibition.

### Example 10

To generate mature cytotoxic T cell (CTL), splenocytes were isolated from OT-I mice and stimulated with 5 μg/ml OVA257-264 (57951-1MG, Sigma) in the presence of 2 ng/ml IL-2 for 3 days. T cells were then centrifuged and cultured in RPMI1640 medium containing of 2 ng/ml murine IL2, 10% FBS and 1% PS. To measure the cytotoxicity of CD8+ T cells, 50,000 enriched CD8+ T cells were mixed in 96-well plates with EO771-OVA cells at ratios of 5:1, 1:1 or 1:5. The drug was added to EO771 cells or CD8+ T cells 48 hours before co-culture. We assessed the killing efficiency by measuring the activity of luciferase (#E1910, Dual-Luciferase® Reporter Assay System, Promega) after 6 hours of co-culture of the T-cells together with the tumor cells.

### Example 11

### Epigenetic Inhibitors Up-Regulate Antigen Processing and Presentation

As BML-210 exhibited the most potent anti-tumor activity in our in vitro and in vivo assays, we wanted to elucidate the molecular mechanism by which BML-210 enhanced the response of breast tumor cells to cytotoxic CD8+ T cells. To this end, we analyzed the genome-wide gene expression profiles to systematically identify transcriptome reprogramming in the BML-210-treated cells. Gene ontology (GO) enrichment analyses of altered gene expression profiles (up-regulated or down-regulated) showed that treatment of BML-210 led to upregulation of genes particularly enriched in lysosome, phagosome, antigen processing and presentation pathways. As examples, MHC-I genes (H2-D1, H2-K2, B2M), lysosome gene (Lamp1), and endosome gene (Hspa8) were induced by the BML-210 treatment. MHC-I-associated antigen processing and presentation pathway in the tumor cell is composed of a number of steps that involves protein degradation and processing, vehicle transport (endosome, lysosome and phagosome and associated cytoskeleton), and antigen loading onto MHC-I complex40,41. Therefore, we reasoned that BML-210 treatment reprograms the gene transcription of tumor cells, leading to increased antigen processing and presentation and enhanced response to the CD8+ T cells. Gene set enrichment of the antigen processing and presentation was performed and the top differentially expressed genes (DEGs) were identified. The positively correlated DEGs were individually confirmed by quantitative PCR (qPCR), showing that most of DEGs had higher expression levels in mouse (EO771) and human (MDA-MB-468) breast cancer cells with BML-210 treatment (FIGS. 4A and 4B). H-2Kb on EO771 cells (FIG. 4C) and HLA on MDA-MB-468 cells (FIG. 4D) were presented at much higher levels when the cells were treated with BML-210, suggesting higher levels of MHC-I-associated antigen presentation. Similarly, the increased H-2Kb or HLA associated antigen presentation was also observed in the cells treated with CUDC-101 and GSK-LSD1, indicating that enhanced antigen presentation is likely a common mechanism for all the three compounds. Confocal images also confirmed that these three compounds boosted up the antigen presentation (FIGS. 4E and 4F) with no other morphological changes observed on the treated cells. Knockdown of B2M in the OVA+ EO771 cells completely abolished the effect of BML-210 on promoting the T cell-mediated cytotoxicity (FIG. 4G). As B2M is an essential component of MHC-1 molecules, this result suggests that BML-210 directly targets the MHC-I-associated antigen presentation pathway and increases the recognition of breast tumor cells by T cells.

### Example 12

To examine whether these three compounds affect other cell types than tumor cells in the tumor organoid screen system, non-tumor cells (GFP− EpCAM−) from the GFP+Luc+OVA+ EO771 tumor organoids treated with each of the three compounds were gated out for analysis. The expression levels of H-2Kb in the GFP−EpCAM− cells did not have any significant changes after drug treatments for 48 h in a dose-gradient analysis. Furthermore, direct treatment of CD8+ T cells with these compounds did not affect the expression levels of GZMB, IFNγ and TNFα at different drug doses (0, 0.01, 0.1, 1 μM). The results suggest that the compounds directly promoted the response of tumor cells to CD8+ T cells in the tumor organoids.

### Example 13

### BML-210 Increases Immune Lymphocyte Infiltration and Enhances PD-1-Based ICB

To examine the BML-210-induced global immunological changes, we harvested mouse mammary tumors 26 days post orthotopic implantation of the EO771 cells for mass cytometry (CyTOF) analysis (FIGS. 5A and 5B and Table 2). The BML-210 treatment led to a notably enhanced infiltration of immune lymphocytes, particularly CD8+ T effector cells and natural killer (NK) cells, but decreased infiltration of polymorphonuclear myeloid-derived suppressor cells (PMN-MDSCs: CD11b⁺Ly6G⁺Ly6ClowF4/80⁻) and increased monocytic myeloid-derived suppressor cells (Mo-MDSCs: CD11b⁺Ly6G-Ly6C⁺F4/80⁻). No significant differences were observed in tumor-associated macrophage (TAM M1: CD1b⁺F4/80⁺MHC-II^{high} and TAM M2: CD11b⁺F4/80⁺MHC-II^{low}) populations. PMN-MDSCs are often immune suppressive, which restrict immune responses and suppress anti-tumor cytotoxicity of T cells. These immunological changes are tipping the balance in the tumor microenvironment towards anti-tumor immunity. Despite the promising potential in cancer therapy, the efficacy of PD-1 blockade in metastatic triple-negative breast cancer is currently low, highlighting a need for strategies that render the tumor microenvironment more sensitive to PD-1 blockade. We reasoned that the enhanced antigen presentation and tumor cell recognition would improve the efficacy of the anti-PD-1-based immunotherapy. It is known that anti-PD-1 therapy modestly enhanced anti-tumor response in mouse EO771 tumor models. Thus, we assessed the therapeutic responses of breast tumors to the combinational treatment of PD-1 monoclonal antibodies (mAb) with BML-210 (20 mg kg-1) in C57BL/6 mice bearing EO771-derived tumors (FIGS. 5C and 5D). The addition of BML-210 to the PD-1 blockade treatment resulted in much greater tumor growth control (indicated by tumor growth inhibition and reduced tumor weight) than BML-210 or PD-1 single agent treatment. The results suggested that BML-210 treatment may promote global antigen presentation of tumor cells regardless of the amount of tumor neoantigens. In addition, due to the immunosuppressive tumor microenvironment, CD8+ T cells are often at inactive or exhausted states, which can be activated by the PD-1 inhibition for better cytotoxicity on the tumor cells with enhanced antigen presentation. Consistent with the results of immune profiling studies (FIGS. 5A and 5B), much higher tumor infiltration of CD8+ T cells was observed in the combinational treatment group, compared to the control group, or the groups with single agent treatment (FIGS. 5E and 5F). Collectively, the results of the mouse tumor models verified the effect of BML-210 in modulating the tumor immune response and rendering the breast tumor sensitive to the ICB therapy.

### Example 14

### Epigenetic Inhibitors Promotes T Cell Activity in Patient-Derived Organoids

There is an unmet clinical need to predict responsiveness to new cancer immunotherapy. Both syngeneic mouse models and humanized patient-derived xenograft (PDX) models are time-consuming and not cost-efficient. Here, we wanted to apply the standardized tumor organoid model in this study to predict the therapeutic responses to BML-210, CUDC-101 and GSK-LSD1. In this setting, autologous CD8+ T cells were isolated from patient tumor tissues and amplified for a week. Patient-derived organoids (PDOs) were generated from 10 patients with breast cancer (Table 2). The PDOs from each patient were characterized and compared with their originating tumors using Hematoxylin and eosin (H&E) staining and immunofluorescence imaging analysis. EpCAM and SMA were used as biomarkers to identify tumor epithelial cells and cancer-associated fibroblast cells, respectively, which are the main components of a breast tumor tissue. Overall, the PDOs exhibited well-organized structure similar to their original tumors. We were able to isolate reasonable numbers (>1×10⁵) of autologous T cells from 8 out of 10 breast tumor tissues. This was expected as previous treatments often result in very low level of immune infiltrate. The PDOs from each tumor were treated with BML-210, CUDC-101 and GSK-LSD1 for 48 hours in advance and then co-cultured with CD8+ T cells amplified from original autologous cells. Among the eight PDOs co-cultured with their autologous T cells, the PDOs from five tumors (PDO1, PDO3, PDO4, PDO5 and PDO8) exhibited significantly higher T cell-mediated cytotoxicity with treatment of BML-210, CUDC-101 or GSK-LSD1. The PDO6 also exhibited higher T cell-mediated cytotoxicity when treated with BML-210 or GSK-LSD1, but showed modest effect with CUDC-101 treatment (FIG. 6). The remaining two PDOs (PDO2 and PDO7) had only moderate increases (statistically not significant) on the T cell cytotoxicity upon the drug treatment. Thus, ˜75% (6 out 8) of the clinical samples showed significant treatment effect of these drugs in the co-culture. Several HDAC inhibitors have been approved for the treatment of patients with hematological malignancies. However, this therapeutic class has yet to be considered an option for patients with breast cancer. Recent studies showed that the patients with hormone receptor-positive (HR+) breast cancer could benefit from treatment with HDAC inhibitors (tucidinostat and vorinostat). HDACi may also upregulate PD-L1 in TNBC cells and thus potentiate immunotherapy, but it was tested in human breast cancer samples. LSD1 ablation by GSK-LSD1 seems to stimulate anti-tumor immunity via activation of type 1 interferon in mouse melanoma models. Our study demonstrate that small molecule compounds for epigenetic modulation are capable to functionally promote antigen processing and presentation on breast tumor cells. Given that immune evasion of tumor cells is a major hurdle in cancer immunotherapy, the treatment of epigenetic inhibitors, such as BML-210, CUDC-101 and GSK-LSD1, can sensitize breast tumors to the immune checkpoint blockade therapy.

### Example 15

Personalized cancer therapy and precision medicine are relied on the study of patient-derived tumor tissues and cells23,49. In recent years, tumor organoids have been emerging as an important tool for predicting clinical responses in cancer therapy. Human clinical samples are hardly used for high-throughput screen due to limited amount, tissue heterogeneity, and high expense1. In this study, we developed a standardized protocol to establish a tumor organoid-T cell system with breast tumor organoids and primary tumor-specific CD8+ T cells. This system is able to facilitate high-throughput drug screens using mouse breast tumor organoids as well as cancer drug prediction using PDOs, because 1) the sufficient source of tumor-specific CD8+ T cells is available for large-scale drug screen; 2) tumor organoids are relatively uniform in size and a pool of tumor organoids used for each drug test offsets the heterogeneity between organoids; 3) tumor-specific T cells or autologous T cells from clinical samples preserve the original T cell receptor spectrum. In this study, cell composition, size and hypoxia of tumor organoids were characterized and optimized for immunotherapy drug screen. First, to avoid interference, immune cells from original tumors were excluded from tumor organoids. Although it is known that immune cells are a part of tumor microenvironment18, they are not essential components of cancer organoids. The complexity of culture condition in vitro and the heterogeneity of immune cells existed in different tumor organoids only lead to inaccurate measurement in drug screen. On the other hand, tumor organoids without immune cells can only be used to screen drugs that promote CD8+ T mediated cytotoxicity, yet this is the aim to be achieved in our study as the CD8+ cytotoxic T lymphocytes are on the center of current cancer immunotherapy. Second, hypoxia signaling can trigger resistance to immune therapy. Hypoxia in large tumor organoids results in massive cell death, suggesting that tumor organoids with size larger than 150 μm is not a good option for drug screen. Indeed, our study showed that tumor organoids with size between 70-150 μm maintain the tumor architecture formed by epithelial, vascular endothelial and stroma cells and inherit the property of tumorigenesis from the original tumor. Finally, the 7-day culture time allows tumor organoids to maintain similar cellular composition as their original tumors, an important feature for drug screen.

From three drug candidates identified from this study, GSK-LSD1 has a confirmed activity as a lysine specific demethylase (LSD1) inhibitor. Inhibition of LSD1 enhanced tumor immunogenicity by stimulating endogenous retrovirus expression and downregulating RNA induced silencing complex in a mouse melanoma model39. Both BML-210 and CUDC-101 are Class I and Class II HDAC inhibitors (HDACis). However, CUDC-101 is possibly an inhibitor of the receptor kinases epidermal growth factor receptor (EGFR) and human epidermal growth factor receptor 2 (HER2). As HDACis have been considered to leverage the efficacy of immunotherapy, we focused on BML-210 as an HDAC inhibitor for molecular mechanism study with gene expression data. Results from immunological studies are in line with our analysis of RNA-seq data in the BML-210-treated cells, suggesting that BML-210 upregulates MHC-I-associated antigen processing and presentation. This mechanism is probably shared by GSK-LSD1 and CUDC-1 because they also promote the MHC-I antigen presentation on breast tumor cells. This study also confirmed the anti-tumor activity of BML-210 in cancer immunotherapy as single agent or in combination with PD-1 checkpoint blockade. The three drugs identified in this study upregulate the antigen processing and presentation machinery at least partly by transcriptional reprogramming in breast tumor cells, which would not be limited to any neoantigens. Given the low response rate in PD-1-based immunotherapy for TNBC, these drugs are potentially great candidates for the combinational therapy with PD-1 or PD-L1 blockade.

In summary, this study provides a repeatable and standardized protocol for immunotherapy drug discovery, in which we characterized and optimized tumor organoids in histological feature, composition, hypoxia, tumorigenesis and culture time, and thus facilitated a large-scale drug screen. The specific recognition between tumor organoids and CD8+ T cells makes the tumor cell killing activity standardized and measurable in a drug screen. PDO models provided solid validation of our screening results. The treatment of BML-210 can sensitize breast tumors to the immune checkpoint blockade therapy, warranting further preclinical studies and potential clinical trials. Application of our tumor organoid-T cell system to different types of tumors such as colorectal and lung cancers will accelerate drug identification for cancer immunotherapy.

1. A method of enhancing the efficacy of CD8+ T cell cytotoxicity against a cancerous cell, or increasing the presentation of antigen-loaded MHC-I complex on a cancer cell, the method comprising contacting said cancerous cell with an epigenetic inhibitor selected from the group consisting of ML-210, CUDC-101 and GSK-LSD1.

2. The method of claim 1 further comprising a step of administering a checkpoint inhibitor in conjunction with the administration of the epigenetic inhibitor.

3. The method claim 2 wherein the checkpoint inhibitor is an inhibitor of cytotoxic T-lymphocyte associated protein 4 (CTLA-4) or programmed death ligand 1 (PD-L1).

4. The method of claim 1 wherein the cancerous cell is a breast cancer cell.

5. (canceled)

6. (canceled)

7. The method of claim 1 wherein the contact occurs in vivo.

8. A method of inhibiting cancer cell growth or cancer cell proliferation, wherein the method comprises contacting a cancer cell with an epigenetic inhibitor selected from BML-210, CUDC-101, or GSK-LSD1 in the presence of T-cells.

9. The method of claim 8 wherein a patient having cancer is administered a pharmaceutical composition comprising an epigenetic inhibitor selected from BML-210, CUDC-101, or GSK-LSD1.

10. The method of claim 9 wherein the cancer is a solid tumor, optionally wherein the cancer is breast cancer.

11. The method of claim 8 further comprising the administration of a checkpoint inhibitor, optionally wherein the checkpoint inhibitor is an inhibitor of cytotoxic T-lymphocyte associated protein 4 (CTLA-4) or programmed death ligand 1 (PD-L1).

12. A method of for identifying compounds for their ability to enhance the sensitivity of tumor cell to T-cell cytotoxicity, said method comprising
generating tumor organoids from cancer cells;
treating said generated tumor organoids that have a diameter between 70 and 150 μm with a candidate drug for 48 h;
contacting the treated tumor organoids with pre-activated CD8+ T cells from mice; and
measuring the release of a intracellular marker from cells of the tumor organoids, to assess the T cell-mediated cytotoxicity effect.

13. The method of claim 12, wherein the candidate drug is an epigenetic inhibitor.

14. The method of claim 13, further comprising contacting the tumor organoids with a checkpoint inhibitor.

15. The method of claim 14 wherein the checkpoint inhibitor is an inhibitor of cytotoxic T-lymphocyte associated protein 4 (CTLA-4) or programmed death ligand 1 (PD-L1).

16. The method of claim 2 wherein the cancerous cell is a breast cancer cell.

17. The method of claim 2 wherein the contact occurs in vivo.


SEQUENCE LISTING (55 sequences)

SEQ ID NO: 1 - DNA, 21 bp, Artificial Sequence
  PCR primer
ttctggtgcttgtctcactga

SEQ ID NO: 2 - DNA, 22 bp, Artificial Sequence
  PCR primer
cagtatgttcggcttcccattc

SEQ ID NO: 3 - DNA, 21 bp, Artificial Sequence
  PCR primer
tggctgctcccaataatgtct

SEQ ID NO: 4 - DNA, 22 bp, Artificial Sequence
  PCR primer
gagggtagtgaccaaaagatgg

SEQ ID NO: 5 - DNA, 19 bp, Artificial Sequence
  PCR primer
ccgtggacttcttcgagcc

SEQ ID NO: 6 - DNA, 23 bp, Artificial Sequence
  PCR primer
ctgttgaatcaaactcaatgggc

SEQ ID NO: 7 - DNA, 19 bp, Artificial Sequence
  PCR primer
ctgacgatgatcgcccgtg

SEQ ID NO: 8 - DNA, 23 bp, Artificial Sequence
  PCR primer
tgcttagcctgactctgatactg

SEQ ID NO: 9 - DNA, 20 bp, Artificial Sequence
  PCR primer
agatggggtccggttcagtt

SEQ ID NO: 10 - DNA, 20 bp, Artificial Sequence
  PCR primer
ggtaggtcgggtctctcctt

SEQ ID NO: 11 - DNA, 23 bp, Artificial Sequence
  PCR primer
tcctgtccacaatacactgatgt

SEQ ID NO: 12 - DNA, 21 bp, Artificial Sequence
  PCR primer
gaaccaccgtagttcgactgt

SEQ ID NO: 13 - DNA, 20 bp, Artificial Sequence
  PCR primer
ctggccggaatgcagatcag

SEQ ID NO: 14 - DNA, 21 bp, Artificial Sequence
  PCR primer
tgaggataggagccgtatgga

SEQ ID NO: 15 - DNA, 23 bp, Artificial Sequence
  PCR primer
ggagaggtctttgttcctttgtt

SEQ ID NO: 16 - DNA, 21 bp, Artificial Sequence
  PCR primer
gtctctgttcttgagcttccc

SEQ ID NO: 17 - DNA, 21 bp, Artificial Sequence
  PCR primer
atggaagcgttttgggagtca

SEQ ID NO: 18 - DNA, 20 bp, Artificial Sequence
  PCR primer
gttctgggtccgagtgatgg

SEQ ID NO: 19 - DNA, 19 bp, Artificial Sequence
  PCR primer
tggacgagacaagacaccc

SEQ ID NO: 20 - DNA, 21 bp, Artificial Sequence
  PCR primer
ctccacgagcatctccagttc

SEQ ID NO: 21 - DNA, 21 bp, Artificial Sequence
  PCR primer
ggcatgtacgagcaactcaag

SEQ ID NO: 22 - DNA, 21 bp, Artificial Sequence
  PCR primer
gctcaagcagaaccaacttca

SEQ ID NO: 23 - DNA, 23 bp, Artificial Sequence
  PCR primer
agcgtcaacaagataagaatgct

SEQ ID NO: 24 - DNA, 22 bp, Artificial Sequence
  PCR primer
gcccgattcctatatgctcaaa

SEQ ID NO: 25 - DNA, 20 bp, Artificial Sequence
  PCR primer
gcaggaggtcgagctatgtt

SEQ ID NO: 26 - DNA, 22 bp, Artificial Sequence
  PCR primer
tgagaacccaaatgcaatgctt

SEQ ID NO: 27 - DNA, 19 bp, Artificial Sequence
  PCR primer
accagcagtacgcctacga

SEQ ID NO: 28 - DNA, 20 bp, Artificial Sequence
  PCR primer
aaccagaacagcaacggtcg

SEQ ID NO: 29 - DNA, 21 bp, Artificial Sequence
  PCR primer
gaggctatccagcgtactcca

SEQ ID NO: 30 - DNA, 21 bp, Artificial Sequence
  PCR primer
cggcaggcatactcatctttt

SEQ ID NO: 31 - DNA, 21 bp, Artificial Sequence
  PCR primer
cctgccgtctacttcaaggag

SEQ ID NO: 32 - DNA, 21 bp, Artificial Sequence
  PCR primer
gaacttgccggaactgagaac

SEQ ID NO: 33 - DNA, 21 bp, Artificial Sequence
  PCR primer
atctgtgaggtcgaaacagga

SEQ ID NO: 34 - DNA, 23 bp, Artificial Sequence
  PCR primer
tggagcatactcaacagtctttg

SEQ ID NO: 35 - DNA, 21 bp, Artificial Sequence
  PCR primer
cgtgacgccagtgaagaatca

SEQ ID NO: 36 - DNA, 21 bp, Artificial Sequence
  PCR primer
cgctcagtgagacaagtttcc

SEQ ID NO: 37 - DNA, 22 bp, Artificial Sequence
  PCR primer
ccgggaccttcaacaatatcag

SEQ ID NO: 38 - DNA, 22 bp, Artificial Sequence
  PCR primer
gcaaaagccaacttcttaggga

SEQ ID NO: 39 - DNA, 23 bp, Artificial Sequence
  PCR primer
ggagtccgatttagttggaatgt

SEQ ID NO: 40 - DNA, 21 bp, Artificial Sequence
  PCR primer
aggtctctctttcagtggtgt

SEQ ID NO: 41 - DNA, 19 bp, Artificial Sequence
  PCR primer
atgtcccagccgggaatac

SEQ ID NO: 42 - DNA, 20 bp, Artificial Sequence
  PCR primer
aggaccgttggtgtagggag

SEQ ID NO: 43 - DNA, 21 bp, Artificial Sequence
  PCR primer
aacgtcaaccagtcagttcca

SEQ ID NO: 44 - DNA, 19 bp, Artificial Sequence
  PCR primer
ggctccatagggaatggcg

SEQ ID NO: 45 - DNA, 21 bp, Artificial Sequence
  PCR primer
gtcaacaaggttacgtggcta

SEQ ID NO: 46 - DNA, 21 bp, Artificial Sequence
  PCR primer
tagtgcccataatgaggtgga

SEQ ID NO: 47 - DNA, 19 bp, Artificial Sequence
  PCR primer
gccgtaaatcaggcggtct

SEQ ID NO: 48 - DNA, 20 bp, Artificial Sequence
  PCR primer
gcccttgagttgctcgtaca

SEQ ID NO: 49 - DNA, 19 bp, Artificial Sequence
  PCR primer
gcctccatcgacatcctcc

SEQ ID NO: 50 - DNA, 20 bp, Artificial Sequence
  PCR primer
gagctgctttagccttgctg

SEQ ID NO: 51 - DNA, 19 bp, Artificial Sequence
  PCR primer
gaagcgtttttggggtcgc

SEQ ID NO: 52 - DNA, 19 bp, Artificial Sequence
  PCR primer
gagtggacggaatgcggta

SEQ ID NO: 53 - DNA, 23 bp, Artificial Sequence
  PCR primer
aaaaggagggagttacactcagg

SEQ ID NO: 54 - DNA, 21 bp, Artificial Sequence
  PCR primer
gctgtgagggacacatcagag

SEQ ID NO: 55 - protein, 8 aa, Artificial Sequence
  Peptide fragment
SIINFEKL